(19) United States
(12) Patent Application Publication
(10) Pub. No.: US 2013/0245206 A1
(43) Pub. Date: Sep. 19, 2013

(54) Bioabsorbable Polymeric Compositions and Medical Devices

(71) Applicant: ORBUSNEICH MEDICAL, INC., Ft. Lauderdale, FL (US)

(72) Inventors: G. Lawrence Thatcher, Chelmsford, MA (US); Robert J. Cottone, Davie, FL (US)

(73) Assignee: OrbusNeich Medical, Inc., Ft. Lauderdale, FL (US)

(21) Appl. No.: 13/861,051

(22) Filed: Apr. 11, 2013

Related U.S. Application Data
(63) Continuation of application No. 12/578,432, filed on Oct. 13, 2009, now abandoned.
(60) Provisional application No. 61/104,718, filed on Oct. 11, 2008.

(51) Int. Cl. A61L 31/04; A61L 29/04; A61L 29/14; A61L 17/12; A61L 24/00; A61L 27/26; A61L 27/58; A61L 26/00; A61L 24/04; A61L 31/14; A61L 28/00

(52) U.S. Cl. CPC A61L 31/041; A61L 31/148; A61L 29/049; A61L 29/148; A61L 17/12; A61L 28/0026; A61L 28/0057; A61L 27/26; A61L 27/58; A61L 26/0052; A61L 26/009; A61L 24/043; A61L 24/0042
USPC 525/439; 525/437

(57) ABSTRACT

The bioabsorbable polymers and compositions of the present invention may be formed into medical devices such as stents that can be crimped onto a catheter system for delivery into a blood vessel. The properties of the bioabsorbable polymers allow for both crimping and expansion of the stent. The crystal properties of the bioabsorbable polymers may change during crimping and/or expansion allowing for improved mechanical properties such as tensile strength and slower degradation kinetics. Typically, bioabsorbable polymers comprise aliphatic polyesters based on lactide backbone such as poly L-lactide, poly D-lactide, poly D,L-lactide, mesolactide, glycolides, lactones, as homopolymers or copolymers, as well as formed in copolymer moieties with co-monomers such as, trimethylene carbonate (TMC) or ε-caprolactone (ECL).

### BACKGROUND OF THE INVENTION

Although the use of bioabsorbable polymers is well known, the development of effective bioabsorbable polymers for medical devices that undergo high stress such as exposure to the pressures of arterial contraction and blood flow represents a major on-going challenge for biomedical scientists. Thus, the development of a bioabsorbable stent that would retain its shape, yet degrade within a reasonable time period without producing a drastic immune response remains an unsolved problem.

Bioabsorbable polymers comprise a wide range of different polymers. Most typically, bioabsorbable polymers are formed from aliphatic polyesters based on a lactide backbone such as, poly L-lactide, poly D-Lactide, poly D,L-Lactide, mesolactide, glycolides, homopolymers, or heteropolymers formed in copolymer moieties with co-monomers such as, trimethylene carbonate (TMC) or ε-caprolactone (ECL). U.S. Pat. No. 6,706,854; U.S. Pat. No. 6,607,548; EP 0401844; WO 2006/111578; and, Jeon et al. Synthesis and Characterization of Poly (L-lactide)—Poly (ε-caprolactone) Multiblock Copolymers. Macromolecules 2003: 36, 5585-5592. Moreover, the use of biodegradable materials with a medical device such as a stent can help to overcome some of the traumatic stress injuries, such as restenosis, that is commonly associated with metal stents.

The synthesis of polylactides is well understood chemically (see, for example, http://www.puracbiomaterials.com/purac_bio_com, Oct. 10, 2009/; http://www.boehringer-ingelheim.com/corporate/ic/pharmachem/products/resomer.asp, Oct. 10, 2009). Once a polymer is formed, it can be blended together with other polymers or pharmaceutical agents, extruded or molded and then, subjected to temperature changes or physical stress. This treatment alters the final crystalline structure resulting in a composite or hybrid material that has unique physical characteristics, including both crystal structures as well as mechanical properties.

The bioabsorbable polymer blends typically include a base polymer (which itself may be a blend) and an additive polymer; the additive polymer imparts additional molecular free volume to the base polymer allowing for sufficient molecular motion of the polymers so that under physiological conditions, re-crystallization can occur. In addition, increased molecular free volume also allows for increased water uptake which facilitates bulk degradation kinetics. This property allows for incorporation of temperature sensitive, pharmaceutically active agents into the blend.

Because inflammation which ultimately results in restenosis represents a major issue with the introduction of any “foreign” medical device such as a metal stent, it is also important to develop polymer blends that will not stimulate the immune system to the extent observed with other medical devices. For example, the enhanced hydrophilicity of certain polymer blends reduces activation of the complement system. (see, Dong et. al, J. of Biomedical Materials Research, part A, DOI 10.1002, 2006).

Thus, developing a polymer blend that will produce a structurally strong medical device such as stent which will remain for a defined period within the body and then degrade without generating a massive immune response is critical.

### SUMMARY OF THE INVENTION

The present invention provides for a composition formed from a blend of polymers, comprising a polymer formed from poly-L-lactide, poly-D-lactide or mixtures thereof and a copolymer moiety comprising poly-L-lactide or poly-D-lactide linked with ε-caprolactone or trimethylcarbonate. The copolymer moiety comprises poly-L-lactide or poly-D-lactide linked with ε-caprolactone or trimethylcarbonate wherein, the poly-L-lactide or poly-D-lactide sequence in the copolymer moiety is random with respect to the distribution of ε-caprolactone or trimethylcarbonate. The wide-angle X-ray scattering (WAXS) exhibits 20 values of about 16.48 and about 18.76. In certain embodiments, the copolymer moiety is poly-L-lactide or poly-D-lactide linked with ε-caprolactone.

In one embodiment, the composition can be made from a blend having about 20% (w/w) to about 45% (w/w) poly-L-lactide, about 30% (w/w) to about 50% (w/w) poly-D-lactide and about 10% (w/w) to about 35% (w/w) poly L-lactide-co-TMC (about 60/40 mole/mole to about 80/20 mole/mole, with about 70/30 mole/mole being one embodiment) or poly-L-lactide-ε-caprolactone; the poly-L-lactide or poly-D-lactide ranges from about 20% (w/w) to about 95% (w/w); from about 50% (w/w) to about 95% (w/w); from about 60% (w/w) to about 95% (w/w); or from about 70% (w/w) to about 80% (w/w).

In another embodiment, the composition comprises a blend formed from a polymer formed from poly-L-lactide, poly-D-lactide or mixtures thereof and a copolymer moiety comprising poly-L-lactide or poly-D-lactide linked with ε-caprolactone or trimethylcarbonate. The poly-L-lactide or poly-D-lactide sequence in the copolymer moiety is random with respect to the distribution of ε-caprolactone or trimethylcarbonate and there is at least about 95% (w/w) amorphous material in the composition. In certain embodiments, the percentage amorphous material is at least about 98% (w/w) or 99% (w/w). In various embodiments, the percent crystallinity of the composition ranges from about 0% (w/w) to about 10% (w/w), from about 20% (w/w) to about 70% (w/w), from about 30% (w/w) to about 60% (w/w) or from about 30% (w/w) to about 60% (w/w).

The composition may also be formed from blend of polymers, comprising a polymer formed from poly-L-lactide, poly-D-lactide or mixtures thereof and a copolymer moiety comprising poly-L-lactide or poly-D-lactide linked with ε-caprolactone or trimethylcarbonate. The poly-L-lactide or poly-D-lactide sequence in the copolymer moiety is random with respect to the distribution of ε-caprolactone or trimethylcarbonate and the wide-angle X-ray scattering (WAXS) exhibits 20 values of about 16.65 and about 18.96. The WAXS 20 values may further comprise peaks at about 12.00, about 14.80, about 20.67, about 22.35, about 23.92, about 24.92, about 29.16 and about 31.28.

Under DSC analysis, the polymer composition may exhibit Tₘ peaks at about 180° C. and about 217° C. or about 178° C. and about 220° C.

### BRIEF DESCRIPTION OF THE DRAWINGS

FIG. 1—DSC P11228 Untreated (Raw) Material

FIG. 2—DSC P11228 annealed at 120° C. for 15 minutes

FIG. 3—DSC P11228 annealed at 120° C. for 15 minutes and stressed

FIG. 4—DSC P11369 Untreated

FIG. 5—DSC P11369 annealed at 80° C. for 15 minutes

FIG. 6—DSC P11369 annealed at 80° C. for 15 minutes and stressed

FIG. 7—DSC P11371 Untreated

FIG. 8—DSC P11371 annealed at 80° C. for 15 minutes

FIG. 9—DSC P11371 annealed at 80° C. for 15 minutes and stressed

FIG. 10—WAXS P11371 Untreated

FIG. 11a—WAXS P11371 annealed at 80° C. for 15 minutes

FIG. 11b—Peak Analysis WAXS P11371 annealed at 80° C. for 15 minutes

FIG. 12a—WAXS P11371 annealed at 80° C. for 15 minutes and stressed

FIG. 12b—Peak Analysis WAXS P11371 annealed at 80° C. for 15 minutes and stressed

FIG. 13—WAXS P11369 Untreated

FIG. 14a—WAXS P11369 annealed at 80° C. for 15 minutes

FIG. 14b—Peak Analysis WAXS P11369 annealed at 80° C. for 15 minutes

FIG. 15a —WAXS P11369 annealed at 80° C. for 15 minutes and stressed

FIG. 15b—Peak Analysis WAXS P11369 annealed at 80° C. for 15 minutes and stressed

FIG. 16—WAXS P11228 Untreated

FIG. 17a—WAXS P11228 annealed at 120° C. for 15 minutes

FIG. 17b—Peak Analysis WAXS P11228 annealed at 120° C. for 15 minutes

FIG. 18a—WAXS P11228 annealed at 120° C. for 15 minutes and stressed

FIG. 18b—Peak Analysis WAXS P11228 annealed at 120° C. for 15 minutes and stressed

FIG. 19a—Elongation P11369

FIG. 19b—Elongation P11371

FIG. 20a—Tensile Strength P11369

FIG. 20b—Tensile Strength P11371

### DETAILED DESCRIPTION OF THE INVENTION

The bioabsorbable polymers and compositions of the present invention may be formed into balloon-expandable stents that can be crimped onto a balloon delivery catheter system for delivery into a blood vessel. Alternatively, the bioabsorbable stents may be self-expanding. The balloon expandable medical device comprises a thermal balloon or a non-thermal balloon. The properties of the bioabsorbable polymers allow for both crimping and expansion of the stent on the balloon catheter without material deformation, such as strut fracture. The crystal properties of the bioabsorbable polymers may change during crimping and/or expansion allowing for improved mechanical properties such as tensile strength, creep and slower degradation kinetics.

During breakdown, the bioabsorbable polymers of the present invention exhibit lower immunogenicity, e.g., decreased IL-2 or other cytokine production, as compared with other bioabsorbable polymers that are seen in the prior art. The in vitro degradation kinetics of the present bioabsorbable polymers show less about 5% overall breakdown after storage for 1 month at physiological conditions (e.g., phosphate buffered saline at 37° C.); in other embodiments, the overall breakdown is less than about 10%, 20%, 30% or 40% after storage for 1 month, 2 months, 3 months or 6 months at physiological conditions. As defined herein, overall breakdown encompasses change in molecular properties, e.g., molecular weight, crystalline properties, mass loss or loss of mechanical properties. When formed into a stent, the bioabsorbable polymers of the present invention retain sufficient mechanical strength to maintain patency of a blood vessel for at least about 1 month, 2 months, 3 months, 4 months, 5 months, 6 months, 1 year, 2 years or 3 years after implantation. The stents of the present invention can be structurally configured to conform to any vessel shape.

Bioabsorbable polymers represent a wide range of different polymers. Typically, bioabsorbable polymers comprise aliphatic polyesters based on lactide backbone such as poly L-lactide, poly D-lactide, poly D,L-lactide, mesolactide, glycolides, lactones, as homopolymers or copolymers, as well as formed in copolymer moieties with co-monomers such as, trimethylene carbonate (TMC) or ε-caprolactone (ECL). U.S. Pat. No. 6,706,854; U.S. Pat. No. 6,607,548; EP 0401844; and Jeon et al. Synthesis and Characterization of Poly (L-lactide)—Poly (ε-caprolactone). Multiblock Copolymers Macromolecules 2003: 36, 5585-5592. The copolymers comprises a moiety such as L-lactide or D-lactide of sufficient length that the copolymer can crystallize and not be completely sterically hindered by the presence of glycolide, polyethylene glycol (PEG), ε-caprolactone, trimethylene carbonate or monomethoxy-terminated PEG (PEG-MME). For example, in certain embodiments greater than, 7, 8, 9, 10, 50, 75, 100, 150 or 250 L or D-lactides may be arrayed sequentially in a polymer. Fukushima et al. Sterocomplexed polylactides (Neo-PLA) as high-performance bio-based polymers: their formation, properties and application. Polymer International 55:626-642 (2006). These blocks of L or D-lactides may allow for cross moiety crystallization even with the addition of an impact modifier to the blend composition. Such a blend makes it possible to design device specific polymer compositions or blends by producing either single or double Tg's (glass transition temperatures). Cross moiety crystallization of compositions with copolymers typically occurs with those blends with copolymers with co-monomer molar ratios ranging from about 50:50 to about 60:40, 99:1, 95:5, 90:10, 88:12, 70:30 or 80:20.

The bioabsorbable polymers of the present invention comprise a wide range of polymer mixtures at different concentrations. For example, the amounts of lactide polymers such as poly L-lactide, poly D-lactide, poly D,L-lactide, poly L-D,L lactide or a blend of any of the foregoing and can range from about 20% (w/w) to about 95% (w/w). Percent weights of each lactide polymer may also range from about 20% (w/w) to about 95% (w/w), 40% (w/w) to about 95% (w/w), from about 50% (w/w) to about 95% (w/w), from about 60% (w/w) to about 95% (w/w), from about 70% (w/w) to about 95% (w/w) or from about 70% (w/w) to about 80% (w/w) of the polymers. The inherent viscosity of the polymers can range from about 1.8 to about 9.0, about 2.0 to about 4.4, or about 2.5 to about 3.8.

In one embodiment, a composition can comprise about 70% (w/w) poly L-lactide having an inherent viscosity (IV) of about 2.5 to about 3.8, mixed with the copolymer moiety such as poly L-lactide-co-trimethylene carbonate (TMC) (about 60/40 mole/mole to about 80/20 mole/mole, with about 70/30 mole/mole being one embodiment) having an IV of about 1.2 to about 1.8, or about 1.4 to about 1.6.

In another embodiment, the polymer formulation comprises a blend having about 70% (w/w) of the triblock poly L-lactide-co-polyethylene glycol (PEG) (99/1 mole/mole) having an IV ranging from about 2.0 to about 4.4 or about 2.5 to about 3.8 which is mixed with the poly L-lactide-co-TMC (about 60/40 mole/mole to about 80/20 mole/mole, with about 70/30 mole/mole being one embodiment) having an IV of about 1.2 to about 1.8 or about 1.4 to about 1.6.

The polymer composition may also comprise a blend having about 70% (w/w) of a diblock, poly L-lactide-co-PEG-MME (monomethyl ethers) (95/5 mole/mole) having an IV ranging from about 2.0 to about 4.4, or about 2.5 to about 3.8, mixed with poly L-lactide-co-TMC (about 60/40 mole/mole to about 80/20 mole/mole, with about 70/30 mole/mole being one embodiment) having an IV ranging from about 1.2 to about 1.8 or about 1.4 to about 1.6. If ε-caprolactone is substituted for TMC in the co-polymer, the IV of the co-polymer ranges from 1.2 to 2.6 (note, this applies to any substitution of TMC with any ε-caprolactone).

In yet another embodiment, the polymer composition comprises a blend having about 20%-45% (w/w) poly-L-lactide, about 35% (w/w) to about 50% (w/w) poly-D-lactide and about 10% (w/w) to about 35% (w/w) poly L-lactide-co-TMC (about 60/40 to about 80/20 mole/mole, with about 70/30 mole/mole being one embodiment) or poly-L-lactide-ε-caprolactone.

Another embodiment may contain about 33% (w/w), 47% (w/w) and about 20% (w/w) or about 40% (w/w), 40% (w/w) and about 20% (w/w) of the respective components: poly-L-lactide, poly-D-lactide, poly L-lactide-co-TMC (about 60/40 to about 80/20 mole/mole, with about 70/30 mole/mole being one embodiment) or poly-L-lactide-ε-caprolactone, respectively.

The co-polymer of the blend which comprises poly-L-lactide-co-TMC or poly-L-lactide-ε-caprolactone can have an IVs ranging from about 0.8-2.6, 1.2-2.6, 1.2-1.8 or 1.4-1.6 (if TMC is substituted for ε-caprolactone, then the IV of the co-polymer may range from about 0.8 to 6.0, 1.2-2.4, 1.4-1.6, 2.0-2.4).

The polymer bends may also comprise copolymer mixtures of poly-L-lactide-ε-caprolactone and poly L-lactide-co-TMC in varying ratios from 10:1 (w/w) to 1:10 (w/w).

The polymer composition and blends of the present invention may allow for the formation of a lactide racemate or stereo-complex crystal structure between the L and D moieties; in certain embodiments, the stero-complex crystal structure may form between an active pharmaceutical ingredient, small molecule, peptide or protein or an excipient. These types of crystals further enhance the mechanical properties of the stent or medical device. The formation of the racemate (stereo complex) crystal structure can result from formulations comprising combinations of: poly L-lactide with poly D-lactide and poly L-lactide-co-TMC; poly D-lactide with poly L-lactide-co-TMC; poly L-lactide with poly D-lactide-co-TMC; poly L-lactide with poly D-lactide with poly D-lactide-co-TMC; poly L-lactide-co-PEG with poly D-lactide-co-TMC; and, poly D-lactide-co-PEG with poly L-lactide-co-TMC, di-block poly D-co-L-lactide with poly L(or D)-lactide-co-TMC and di-block poly D-co-L-lactide with poly L(or D)-lactide-co-TMC (in each case shown above, ε-caprolactone may be substituted for TMC).

When crystallized from the melt or from solution, homogeneous solutions of poly-L-lactide or poly-D-lactide adopt left- or right-handed 10₃ helix conformations, respectively, and produce the R crystal form by arranging by pair in a crystalline unit cell. The β crystal form, which is only found in solution-spun fibers drawn at high temperatures, features six 3₁ helices in an orthorhombic unit cell and can rearrange to the more stable R crystal form. When crystallized from the melt or from solution, blends of poly-L-lactide and poly-D-lactide can form a racemic sterocomplex. The melting point of this complex (230° C.) is 50° C. higher than that of the R crystal form of the pure polyenantiomers. Brochu et al. Sterocomplexation and Morphology of Polylactides. Macromolecules:5230-5239 (1995). Polymers blends may also form an amorphous mixture. U.S. Pat. No. 6,794,485. The percentage crystallinity may be determined by Differential Scanning calorimetry (DSC). Sarasua, et al. Crystallinity and mechanical properties of optically pure polylactides and their blends, Polymer Engineering and Science: 745-753 (2005).

Poly-lactide racemate compositions also offer the ability to be “cold formable or bendable” without adding heat which can be important if the polymer blend incorporates a pharmaceutical agent which is susceptible to denaturation. Cold-bendable scaffolds of the invention do not require heating to become flexible enough to be crimped onto a carrier device or to accommodate irregularly shaped organ spaces. Cold-formable, includes physiological and ambient temperatures ranging from about 15° C. to about 37.5° C. When implanted in an organ space such as pulsating vascular lumen, cold-bendable scaffolds can afford sufficient flexibility for an expanded scaffold device. For example, in terms of a stent, in certain embodiments, it is desirable to utilize polymeric compositions that possess significant amount of amorphous polymer moieties after fabrication and crystallize when the scaffold is strained by crimping onto a delivery balloon or by stretching upon balloon expansion for implantation. Such cold-bendable polymeric scaffold embodiments do not need to be preheated to a flexible state prior to implantation onto a contoured surface space in the body. Cold-bendability also allows these polymer blends to be both crimped and expanded at physiological and ambient temperature without crazing. Martins et al. Control the Strain-Induced Crystallization of Polyethylene Terephthalate by Temporally Varying Deformation Rates: A Mechano-optical Study. Polymer. 2007: 48, 2109-2123.

Other examples of bioabsorbable polymers that may be used with the methods of the present invention include, aliphatic polyesters, bioglass cellulose, chitin collagen copolymers of glycolide, copolymers of lactide, elastin, tropoelastin, fibrin, glycolide/1-lactide copolymers (PGA/PLLA), glycolide/trimethylene carbonate copolymers (PGA/TMC), hydrogel lactide/tetramethylglycolide copolymers, lactide/trimethylene carbonate copolymers, lactide/-ε-caprolactone copolymers, lactide-σ-valerolactone copolymers, L-lactide/d1-lactide copolymers, methyl methacrylate-N-vinyl pyrrolidone copolymers, modified proteins, nylon-2 PHBA/γ-hydroxyvalerate copolymers (PHBA/HVA), PLA/polyethylene oxide copolymers, PLA-polyethylene oxide (PELA), poly (amino acids), poly (trimethylene carbonates), poly hydroxyalkanoate polymers (PHA), poly(alklyene oxalates), poly(butylene diglycolate), poly(hydroxy butyrate) (PHB), poly(n-vinyl pyrrolidone), poly(ortho esters), polyalkyl-2-cyanoacrylates, polyanhydrides, polycyanoacrylates, polydepsipeptides, polydihydropyrans, poly-d1-lactide (PDLLA), polyesteramides, polyesters of oxalic acid, polyglycolide (PGA), polyiminocarbonates, polylactides (PLA), polyorthoesters, poly-p-dioxanone (PDO), polypeptides, polyphosphazenes, polysaccharides, polyurethanes (PU), polyvinyl alcohol (PVA), poly-β-hydroxypropionate (PHPA), poly-β-hydroxybutyrate (PBA), poly-σ-valerolactone, poly-β-alkanoic acids, poly-β-malic acid (PMLA), poly-ε-caprolactone (PCL), pseudo-Poly(Amino Acids), starch, trimethylene carbonate (TMC) and tyrosine based polymers. U.S. Pat. No. 7,378,144.

Pharmaceutical compositions may be blended into the polymers or may be coated on the polymer blends by spraying, dipping or painting. U.S. Publication Nos. 2006/0172983 A1, 2006/0173065 A1, 2006/188547 A1, 2007/129787 A1. Alternatively, the pharmaceutical compositions may be microencapsulated and then blended into the polymers. U.S. Pat. No. 6,020,385. If the pharmaceutical compositions are covalently bound to the polymer blend, they may be linked by hetero- or homo-bifunctional cross linking agents to the monomer or polymer (see, http ://www.piercenet.com/products/browse.cfm?fldID=020306). It is understood that the polymer blends having pharmaceutical compositions blended, coated or attached may be prepared without undue experimentation.

The pharmaceutical compositions can include (i) pharmacological agents such as, (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, thymidine kinase inhibitors, rapamycin, 40-0-(2-Hydroxyethyl)rapamycin (everolimus), 40-0-Benzyl-rapamycin, 40-0(4′-Hydroxymethyl)benzyl-rapamycin, 40-0-[4′-(1,2-Dihydroxyethyl)]benzyl-rapamycin, 40-Allyl-rapamycin, 40-0-[3′-(2,2-Dimethyl-1,3-dioxolan-4(S)-yl-prop-2′-en-1′-yl]-20 rapamycin, (2′:E,4′S)-40-0-(4′,5′.:Dihydroxypent-2′-en-1′-yl),r apamycin 40-0(2Hydroxy) ethoxycar-bonylmethyl-rapamycin, 40-0-(3-Hydroxypropyl-rapamycin 40-0-((Hydroxy)hexyl-rapamycin 40-0-[2-(2-Hydroxy)ethoxy]ethyl-rapamycin, 40-0-[(3S)-2,2Dimethyldioxolan-3-yl]methyl-rapamycin, 40-0-[(2S)-2,3- Dihydroxyprop-1-yl]-rapamycin, 40-0-(2-Acctoxy)ethyl-rapamycin, 40-0-(2-Nicotinoyloxy)ethyl-rapamycin, 40-0-[2-(N-25 Morpholino) acetoxyethyl-rapamycin, 40-0-(2-N-Imidazolylacetoxy)ethyl-rapamycin, 40-0[2-(N-Methyl-N′-piperazinyl)acetoxy]ethyl-rapamycin, 39-0-Desmethyl-3.9,40-0,0 ethylene-rapamycin, (26R)-26-Dihydro-40-0-(2-hydroxy)ethyl-rapamycin, 28-0 Methyrapamycin, 40-0-(2-Aminoethyl)-rapamycin, 40-0-(2-Acetaminoethyl)-rapamycin 40-0(2-Nicotinamidoethyl)-rapamycin, 40-0-(2-(N-Methyl-imidazo-2′ ylcarbahoxamido)ethyl)-30 rapamycin, 40-0-(2-Ethoxycarbonylaminoethyl)-rapamycin, 40-0-(2-Tolylsulfonamidoethyl)-rapamycin, 40-0-[2-(4′,5′-Dicarboethoxy-1′,2′;3′-triazol-1′-yl)-ethyl]rapamycin, 42-Epi-(telrazolyl)rapamycin (tacrolimus), and 42-[3-hydroxy-2-(hydrox ymethyl)-2-methylpropanoate ]rapamycin (temsirolimus) (WO2008/086369); (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promoters; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e. g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; and, (o) agents that interfere with endogenous vasoactive mechanisms, (ii) genetic therapeutic agents include anti-sense DNA and RNA as well as DNA coding for (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor a and P, platelet-derived endothelial growth factor, platelet-derived growth factor, tumor necrosis factor a, hepatocyte growth factor and insulin-like growth factor, (d) cell cycle inhibitors including CD inhibitors, and (e) thymidine kinase (“TK”) and other agents useful for interfering with cell proliferation.

Other pharmaceutical agents that may be incorporated into the polymer blends, include, acarbose, antigens, beta-receptor blockers, non-steroidal antiinflammatory drugs (NSAID;, cardiac glycosides, acetylsalicylic acid, virustatics, aclarubicin, acyclovir, cisplatin, actinomycin, alpha- and beta-sympatomimetics, (dmeprazole, allopurinol, alprostadil, prostaglandins, amantadine, ambroxol, amlodipine, methotrexate, S-aminosalicylic acid, amitriptyline, amoxicillin, anastrozole, atenolol, azathioprine, balsalazide, beclomcthasone, betahistine, bezafibrate, bicalutamide, diazepam and diazepam derivatives, budesonide, bufexamac, buprcnorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidinc, cotrimoxazole, codeine, caffeine, vitamin D and derivatives of vitamin D, colestyramine, cromoglicic acid, coumarin and coumarin derivatives, cysteine, cytarabine, cyclophosphamide, cyclosporin, cyproterone, cytabarine, dapiprazole, desogestrel, desonide, dihydralazine, diltiazem, ergot alkaloids, dimenhydrinate, dimethyl sulphoxide, dimeticone, domperidone and domperidan derivatives, dopamine, doxazosin, doxorubizin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafmil, orlistat, peptide antibiotics, phenytoin, riluzoles, risedronate, sildenafil, topiramatc, macrolide antibiotics, oestrogen and oestrogen derivatives, progestogen and progestogen derivatives, testosterone and testosterone derivatives, androgen and androgen derivatives, ethenzamide, etofenamate, ctofibrate, fcnofibrate, etofyne, etoposide, famciclovir, famotidine, felodipine, fenoftbrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mcpindolol, meprobamate, meropenem, mesalazinc, mesuximide, metamizole, metformin, methotrexate, methylphenidate, methylpredniso lone, metixene, metoclopramide, metoprolol, metronidazole, mianserin, miconazole, minocycline, minoxidil, misoprostol, mitomycin, mizolastinc, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenytoin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tctracyclins, teryzoline, theobromine, theophylline, butizine, thiamazole, phenothiazines, thiotepa, tiagabine, tiapride, propionic acid derivatives, ticlopidine, timolol, tinidazole, tioconazole, tioguanine, tioxolone, tiropramide, tizanidine, tolazolinc, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobutcrol, tyramine, tyrothricin, urapidil, ursodeoxycholic acid, chenodeoxycholic acid, valaciclovir, valproic acid, vancomycin, vecuronium chloride, Viagra, venlafaxine, verapamil, vidarabine, vigabatrin, viloazine, vinblastine, vincamine, vincristine, vindesine, vinorclbinc, vinpocetine, viquidil, warfarin, xantinol nicotinate, xipamide, zafirlukast, zalcitabine, zidovudine, zolmitriptan, Zolpidem, zoplicone, zotipine and the like. See, e.g., U.S. Pat. No. 6,897,205; see also, U.S. Pat. No. 6,838,528; U.S. Pat. No. 6,497,729.

The medical device can comprise any medical device for implantation including stents, coverings for electrodes, catheters, leads, implantable pacemaker, cardioverter or defibrillator housings, dural closures or sutures, spine cages, joints, screws, rods, ophthalmic implants, femoral pins, hip replacements, bone plates, grafts such as bone graft containment devices, graft fixation, anastomotic devices, perivascular wraps, sutures, staples, shunts for hydrocephalus, dialysis grafts, colostomy bag attachment devices, drainage tubes, leads for pace makers and implantable cardioverters and defibrillators, vertebral disks, bone pins, suture anchors, hemostatic barriers, clamps, screws, plates, clips, vascular implants, tissue adhesives and sealants, tissue scaffolds, various types of dressings (e.g., wound dressings), bone substitutes, intraluminal devices, vascular supports, etc.

In one embodiment, the medical device comprises a stent that is structurally configured to expand in situ when deployed into an artery or a vein and to conform to the blood vessel lumen to reestablish blood flow at the site of injury. The stent can be configured to have many different arrangements so that it is crimpable before deployment and expandable at physiological conditions once deployed. The medical device of present invention includes various embodiments of biodegradable polymeric stents, and/or stent walls with different configuration. U.S. Pat. Nos. 6,117,165, 7,108,714 and 7,329,277 represent several examples of such stents. The stent may be a tubular structure comprising struts designed to allow blood to traverse its walls so that the adjacent tissues are bathed or come in contact with it as blood flows through the area. The particular stent design depends on the size of the stent both radially and longitudinally.

The present invention also provides for methods of making a bioabsorbable polymeric implant comprising: blending a crystallizable polymer composition which comprises a base polymer of poly L-lactide and/or poly D-lactide linked with modifying copolymers comprising poly L (or D)-lactide-co-TMC or poly L(or D)-lactide-co-ε-caprolactone in the form of block copolymers or as blocky random copolymers where the lactide chain length is sufficiently long enough to allow cross-moiety crystallization together with poly-L-lactide or poly-D-lactide polymers at various concentrations; molding, extruding or casting the polymer composition to structurally configure an implant such as a stent; and cutting the implant to form desired patterns. In various embodiments greater than, 7, 8, 9, 10, 50, 75, 100, 150 or 250 L or D-lactides may be arrayed sequentially in a polymer. Fukushima et al. Sterocomplexed polylactides (Neo-PLA) as high-performance bio-based polymers: their formation, properties and application. Polymer International 55:626-642 (2006).

Polymerization reactions are well known to one skilled in the synthesis of polymers. Its principles, applications, and techniques such as initiation and molecular weight control for the polymerization reactions, can be found in George Odian, Principles of Polymerization, 4^{th} Ed ©2004 Wiley-Interscience. The polymers, poly-L-lactide and poly-D-lactide may be prepared by polymerization of the corresponding monomers. The most commonly used catalyst is stannous octoate, but other catalysts such as dibutyl tin(IV) and tin(II) chloride can also be employed. The polymerization reactions can also be initiated with an initiator, for example, ethylene glycol or a long chain alcohol. The reaction can be carried out as fusion polymerization, bulk polymerization, or any other polymerization technology known to a person of skill in the art. The synthesis of the polymers is disclosed in U.S. Pat. Nos. 6,706,854, 6,607,548, EP 0401844WO2003/057756 and WO 2006/111578. Jeon et al. Synthesis and Characterization of Poly (L-lactide)—Poly (ε-caprolactone) Multiblock Copolymers. Macromolecules 2003: 36, 5585-5592.The synthesis of Poly-L-lactide-co-ε-caprolactone is also disclosed in Macromolecules 2003: 36, 5585-5592. In addition, the polymers are available commercially. Vendors include, http://www.purac.com, http://www.boehringer-ingelheim.com/corporate/home/home.asp, www.lakeshorebio.com and http://www.absorbables.com. The range of IV for the polymers includes about 1.2 to about 4.4, about 1.2 to about 1.8, about 2.0 to about 4.4 and about 2.5 to about 3.8. In certain embodiments, polymers with IV less than about 2.0 and greater than about 4.5 may be used.

For example, poly-L-lactide of the desired molecular weight is synthesized from the lactide monomer by ring-opening polymerization. L-lactide (1 mol), stannous octoate [5 mmol, monomer/catalyst ratio (M/C)) 200] and 1,6-hexanediol (25 mmol) are weighed into a round-bottomed flask equipped with a mechanical stirrer. The product is dissolved in chloroform and micro filtered through a 0.45 μm pore membrane filter. The polymer is precipitated by pouring the polymer solution into an excess of methanol, filtered, and dried under vacuum. It is a known technique in the art that reaction conditions, such as M/C, reaction temperature and reaction time, can be modified to control the molecular weight of the poly-L-lactide. Though the preferred catalyst is stannous octoate, other catalysts such as tin(II) chloride or initiator such as ethylene glycol can also be employed. The Tm of the poly-L-lactide polymer typically ranges from about 160° C. to about 194° C. and the IV from about 2.0 to about 4.4 (see, for example, U.S. Pat. Nos. 6,706,854, 6,607,548, EP 0401844WO2003/057756 and WO 2006/111578).

Poly-D-lactide of desired molecular weight may be synthesized from the lactide monomer by ring-opening polymerization. D-lactide (1 mol), stannous octoate [5 mmol, monomer/catalyst ratio (M/C)) 200], and 1,6-hexanediol (25 mmol) are weighed into a round-bottom flask equipped with a mechanical stirrer. The flask is purged with dry nitrogen and immersed in an oil bath at 130° C. for 5 h. The product is dissolved in chloroform and microfiltered through a 0.45 μm pore membrane filter. The polymer is precipitated by pouring the polymer solution into an excess of methanol, filtered, and dried under vacuum. It is a known technique in the art that reaction conditions, such as M/C, reaction temperature and reaction time, can be modified to control the molecular weight of the poly-D-lactide. The preferred catalyst is stannous octoate, but other catalysts such as tin(II) chloride or initiator such as ethylene glycol can also be employed. The Tₘ of the poly-D-lactide polymer typically ranges from about 160° C. to about 194° C. and the IV from about 2.0 to about 4.4.

Random Copolymers moieties are synthesized from the D- or L-lactide and ε-caprolactone monomers by ring-opening polymerization. U.S. Pat. Nos. 6,197,320, 6,462,169, 6,794,485. Caprolactone (100 mmol), D- or L-lactide (100 mmol), stannous octoate (1 mmol), and 1,6-hexanediol (0.5 mmol) are weighed into a glass ampule equipped with a magnetic stirring bar. The ampule is sealed under vacuum after purging three times with nitrogen at 90° C. and heated to 150° C. in an oil bath for 24 h with stirring. After reaction, the ampule is broken; the polymer is then dissolved in chloroform and microfiltered through a 0.45 μm pore membrane filter. It is precipitated by pouring the polymer solution into an excess of methanol, filtered, and dried under vacuum. By controlling the reaction conditions, such as lactide/ε-caprolactone ratio, monomer/catalyst ration, reaction temperature and reaction time, the molecular weight of the copolymer moiety is controlled. The preferred catalyst is stannous octoate; however, other catalysts such as tin(II) chloride or initiator or ethylene glycol can be employed. By controlling the molar ratios of the D- or L-lactides, the number of L-lactides arrayed in sequence in the random copolymer moiety can be controlled, which may range from 10-20, 20-30, 30-40, 40- -50, 100-150 or from 150-200. (see, for example, EP 1468035 B1, U.S. Pat. No. 6,706,854, WO 2006/111578 A1 and WO 03057756 A1). TMC may be substituted for E-caprolactone in the above synthesis procedures.

In various embodiments, di-block copolymers containing poly-L-Lactide and poly-D-Lactide may be used. The use of a di-block copolymer of L- and D-lactide during polymer mixture blending can enhance the formation of the racemate crystal structure having both D- and L-lactides over homo-enantiomer co-crystallization.

During synthesis of the lactide polymers, monomers may be extracted from the reaction by either driving the reactions to “completion” and/or use of known extraction techniques such as solvent extraction or supercritical CO₂ extraction. U.S. Pat. No. 5,670,614.

Polymers used for controlled drug delivery must be biocompatible and degrade uniformly into non-toxic molecules that are non-mutagenic, non-cytotoxic and non-inflammatory. Examples of polyanhydrides and polyesters that are useful in the preparation of the present polymer blends include polymers and copolymers of lactic acid, glycolic acid, hydroxybutyric acid, mandelic acid, caprolactone, sebacic acid, 1,3-bis(p-carboxyphenoxy)propane (CPP), bis-(p-carboxyphenoxy)methane, dodecandioic acid (DD), isophthalic acid (ISO), terephthalic acid, adipic acid, fumaric acid, azeleic acid, pimelic acid, suberic acid (octanedioic acid), itaconic acid, biphenyl-4,4′-dicarboxylic acid and benzophenone-4,4′-dicarboxylic acid. Polymers may be aromatic, aliphatic, hydrophilic or hydrophobic.

The polymer blends are formed using known methods such as solvent mixing or melt mixing. In the solvent mixing procedure, the desired weight of each of the polymers to be blended is mixed in the desired amount of an appropriate organic solvent or mixture of solvents and the polymer solutions mixed. The organic solvent is then removed, for example, by evaporation, leaving a polymer blend residue. Pharmaceutically active agents or additives may be incorporated into the polymer blends by dissolving or dispersing the pharmaceutically active agent or additive in the blend solution prior to removal of the solvent. This method is especially useful for the preparation of polymer blends incorporating pharmaceutically active agents that are sensitive to elevated temperatures.

In the melt mixing procedure, the polymers are melted together or brought separately to each polymer's respective melting temperature and then mixed with each other for a defined time period, e.g., from about two to about thirty minutes (5, 10, 15, 20 and 25 minutes). The blend is then allowed to cool to room temperature. Pharmaceutically active agents or additives may be incorporated by dissolving or dispersing them either in the blend solution or in the individual melt solutions prior to blending. U.S. Patent Publication No. 2006/0172983.

The glass transition temperature (T_{g}), crystallization temperature (T_{c}) and melting temperature (Tₘ) are critical characteristics of the polymer blend. The miscibility of the blended polymers is indicated by a single glass transition temperature (T_{g}) of the blend (either shifted or broadened from the constituents of the blend). A blend with two or more T_{g} indicates degrees of immiscibility of the polymers. The polymer blend may also present no melting temperature (Tₘ) indicating an amorphous polymer blend or single or multiple melting temperatures. Multiple melting temperatures indicate crystalline polymer where the crystals are either single or multiple homo-enantiomer, or co-moiety crystals such as the stereocomplex or racemate crystal structure between poly-L and poly-D-lactides. The present invention comprises a polymorphic polymer system having varying degrees of miscibility (and thus domain size) which affects both mechanical properties and degradation kinetics.

The molecular weight or viscosity of the polymer blend is typically an average of the molecular weights and viscosities of the component polymers. The polymers can be blended together using melt kneading such as a two-roll mill, a Banbury mixer, a single-screw, twin-screw extruder, intermeshing co-rotating screw extruders and multiscrew extruders. Chris Rauwendaal. Mixing in Polymer Processing. Wiley, 1993; http://www.rauwendaal.com/; www.randcastle.com. The polymer blend may also be processed by sheet extrusion, profile extrusion, blown film extrusion, blow molding, rotational molding, thermoform processing, compression molding, transfer molding or injection molding. www.me.gatech.edu/jonathan.colton/me4210/polymer.pdf.

In one embodiment, poly-L-lactide, poly-D-lactide and poly-L-lactide-co-TMC (or ε-caprolactone) are dry-blended together. Raw material components are dry-blended in a multi-axial Turbula type blender under dry N₂ after each component has been dried. The dry-blend is then fed into an extruder or injection molding machine. Alternatively, the dried components may be individually metered into the extruder or molding machine. After extrusion, the polymer blend is processed at temperatures ranging from their T_{g} (glass transition temperature) to above the Tₘ of the racemate.

During mixing in the extruder or molding machine, the polymer components soften and/or melt, then flow in the extruder or molding machine plasticating unit. They may be visualized as independent melt domains until action of the plasticating screw(s) causes intimate mixing by application of both shear and extensional flows. This forced intimacy between the lactide enantiomers allows for formation of a racemate crystal structure. Because of the high Molecular weights, racemate gels can form in this melt at temperatures above the Tₘ of the enantiomers, i.e., 180° C. but below the Tₘ of the racemate 230° C. Racemate crystallization begins at about 195° C., necessitating higher melt temperatures possibly exceeding the Tm of the racemate and/or additional mixing and melt extension. The Tₘ of the poly-L-lactide/poly-D-lactide racemate of the present invention typically ranges from about 195° C. to about 235° C. Brochu et al. Sterocomplexation and Morphology of Polylactides. Macromolecules 1995 28:5230.

The polymer blend may also be melt cast or transferred to a compression mold (transfer mold). The polymer may be molded or extruded to form a finished device. Alternatively, the polymer blend could be solution or gel cast. In solution or gel casting, during removal of the solvent phase, crystallization occurs in the polymer blend. By controlling the solvent removal rate, inter-moiety crystallization may be controlled. The solvent cast films or tubes can undergo further isothermal recrystallization thermal treatment. In melt processes, by introducing a high degree of mixing in the melt and by enhancing this temperature above the Tₘ of the enantiomers, stereocomplex formation of high Mw Poly-lactides crystals is enhanced. Brochu et al. Sterocomplexation and Morphology of Polylactides. Macromolecules 1995 28:5230. Finished or semi-finished devices or components may undergo further isothermal recrystallization thermal treatment.

The polymer compositions may be prepared from commercially available granular materials and copolymer additives. In one embodiment, the dry components are weighed according to the desired weight ratio into a container rotating for 30 minutes or until a homogenous mixture is obtained, and may be followed by further drying, for example, in a vacuum at 60° C. for 8-12 hours or overnight. The thoroughly mixed components may be melt blended and injection molded into a pair of matching plates. The composition may be extruded at a melt temperature 185-250° C. using a screw with a length to diameter ratio ranging from 16 to 32/1 or 24-26/1 at 2-100 rpm. The polymer blends may be extruded to form, for example, tubes, sheets or fibers. The tubes may be cut into stents or sheets. Additionally, the sheets of fibers may be cut and fabricated into stents.

Stents form scaffolds that may be used in angioplasty. The stents are positioned in narrowed vessel lumens to support the vessel walls. Placement of a stent in the affected arterial segment prevents elastic recoil and closing of the artery. Stents also prevent local dissection of the artery along the medial layer of the artery. Stents may be used inside the lumen of any physiological space or potential space, such as an artery, vein, bile duct, urinary tract, alimentary tract, tracheobronchial tree, cerebral aqueduct or genitourinary system. Stents may also be placed inside the lumen of human as well as non-human animals. In general there are two types of stents: self-expanding and balloon-expandable. The balloon-expandable stent is placed in a diseased segment of a vessel by inserting a crimped stent into the affected area within the vessel. The stent is expanded by positioning a balloon inside the stent. The balloon is then inflated to expand the stent. Inflation remodels the arterial plaque and secures the stent within the affected vessel.

In contrast, a self-expanding stent is capable of expanding by itself. There are many different designs of self-expanding stents, including, coil (spiral), circular, cylinder, roll, stepped pipe, high-order coil, cage or mesh. U.S. Pat. No. 6,013,854. The self-expanding stent is placed in the vessel by inserting the stent in a constrained state into the affected region, e.g., an area of stenosis. Once the constraining sheath is wthdrawn, the stent freely expands to a preset diameter. The stent may be compressed using a tube that has a smaller outside diameter than the inner diameter of the affected vessel region. When the stent is released from confinement in the tube, the stent expands to resume its original shape and becomes securely fixed inside the vessel against the vessel wall.

The stent is formed from a hollow tube made of bioabsorbable polymer. Notches or holes are made in the tube forming the elements of the stent. The notches and holes can be formed in the tube by use of a laser, e.g., UV Eximer lasers” or “Femtosecond lasers”. High-repetition-rate low-pulse-energy near-infrared femtosecond laser pulses from a Ti:sapphire oscillator may be used to micromachine localized refractive index structures inside polymers. The formation of the notches and holes to prepare the claimed stent is considered within the knowledge of a person of ordinary skill in the art. The polymer blends may also be injection molded to a finished or semi-finished shape. Yoklavich et al. Vessel Healing Response to Bioaborbable Implant. Fifth World Biomaterials Congress. May 29-Jun. 2, 1996, Toronto, Canada.

To facilitate placement of the stent within the patient, electron-dense or x-ray refractile markers may be mixed with the polymeric material prior to blending. Radiopaque compounds can be selected from x-radiation dense or refractile compounds such as metal particles or salts. Suitable marker metals may include iron, gold, colloidal silver, zinc, magnesium, either in pure form or as organic compounds, tantalum, tungsten, platinum/iridium, platinum or radioopaque ceramics such as zirconium oxide. To achieve proper blend of marker material a solvent system may include two or more acetone, toluene, methylbenzene, DMSO.

The physical parameters of the polymer mixture can be characterized using a variety of different methods. The following list is nonexhaustive and other methodologies may also be utilized. The molecular weight and distribution of the polymers can be measured by gel permeation chromatography (GPC) or size exclusion chromatography (SEC) (e.g., Waters HPLC systems 410 differential refractometer, three PLGel columns (HR2, HR4, and HR5E), 515 pump). Average molecular weight (Mw), the number average molecular weight (Mn) and molecular weight distribution may be determined by GPC. “Molecular weight distribution” refers to Mw divided by Mn. One could also use dilute solution viscometry to measure intrinsic viscosity which can be correlated to molecular weight of the polymers (see, for example, www.boehringer-ingelheim.com/.../ic/.../N02-06_IV_vs_SEC..pdf, Oct. 10, 2009).

Differential scanning calorimetry (DSC) may be used to study the thermal properties, degree of crystallinity and stereocomplexation of the present compositions. In one embodiment, the result of a DSC measurement using a Differential Scanning calorimeter is a curve of heat flux versus temperature. Examples of the properties of the polymer that may be obtained using DSC include glass transition temperatures (T_{g}), crystallization temperature (T_{c}) and melting temperature (Tₘ). DSC may also be used to examine the purity and composition of the polymer. The crystallinity of the present polymer compositions may range from about 0% to about 10%, about 10% to about 20%, about 20% to about 70%, about 20% to about 40%, about 30% to about 60%, or from about 40% to about 50% (all values are weight/weight (w/w)).

Wide-angle X-ray scattering (WAXS) or small-angle X-ray scattering (SAXS) may be used to determine the crystalline structure, degree of crystallinity and stereocomplexation of the polymer (http://ww-w.panalytical.com/index.cfm?pid=143). In one embodiment, the sample is scanned in a wide angle X-ray goniometer, and the scattering intensity is plotted as a function of the 2θ angle. Tsuji, Poly(lactide) Sterocomplexes, Formation, Structure, Properties, Degradation and Applications. Macro. Mol. Bio. Sci. 5:569-597 (2005).

The morphology of the present polymer may be studied by scanning electron microscopy (SEM) or transmission electron microscopy (TEM). In one embodiment, a polymer sample is sputter-coated with gold layer using a sputter-coater before mounted on the microscope. For the degradation test in vitro, the appearance of pores, cracks, channels or other similar structure may indicate the ongoing erosion of the polymer.

The morphology of the present polymer may also be determined by polarized light microscopy, atomic force microscopy (AFM) or energy dispersive X-ray spectroscopy (EDS). In one embodiment, a polarizing optical microscope equipped with a heating device is used. The sample is placed on a glass plate, heated to its melting temperature (Tm), and then cooled at 10° C./min to 120° C.

The chemical compositions of the present polymer may be identified by Infrared (IR) or Raman spectroscopy. The chemical composition, copolymer and blend ratio and end groups of the present polymers may be studied by magnetic resonance spectroscopy (NMR). In one embodiment, ¹H-NMR spectrum of the polymer is recorded in CDCl₃. In another embodiment, ¹³C-NMR spectrum of the polymer is recorded. The inherent viscosity and molecular weight of a polymer may be determined by viscometry.

The molecular weight of the present polymer may also be determined by static light scattering (SLS). The thermal stability of the present polymer may be determined by thermogravimetric analysis (TGA) and the surface chemical composition of the present polymer may be studied by X-ray photoelectron spectroscopy (XPS). The melt viscosity and stress relaxation of the present polymers may be determined by rheology.

Mechanical properties of the polymers may be assessed. For example, Tensile testing can be performed using an Instron testing machine that elongates a sample, where the force required to break the sample is recorded. This produces a stress strain curve from which mechanical properties (modulus, strength, yield and elongation at break) are measured. Compression testing can also be measured using an Instron testing machine that places a sample under a crushing load and deformation is recorded. Flexural testing may be performed using an Instron testing machine or dynamic materials analysis that places a sample in a three-point bending apparatus to record the stiffness of a material. In this assay, flexural strength and flexural modulus are recorded. Dynamic mechanical analysis (DMA) is used to measure thermal transitions and mechanical properties of polymers resulting from changes in temperature, time, frequency, force, and strain placed on a sample. Density can also be assessed by Gas Pycnometer. http://www.polymathiclabs.com/mechanical_physical.php.

Strain induced crystallization will also be examined. Uniaxial and biaxial deformations as well as the post annealing stage affect the development of structure and performance characteristics. The crystal structures and physical parameters of the polymer compositions are measured during deformation at all stages. X-ray diffraction techniques, on-line spectral bi-refringence techniques, real time FTIR, RAMAN spectroscopy and PET may be used to monitor crystallinity. Martins et al. Polymer 48: 2109-2123 (2007).

Many polymers display another type of localized yielding behavior which results in whitening of the polymer in the region of maximum deformation. Under a microscope, these localized regions of yielding display an increase in volume (dilatation) through formation of micro-cracks which are bridged by polymer fibrils. Crazing and stress whitening are the typical deformation mechanisms. Because crazing is a dilatational mechanism it is expected to occur in regions of high dilatational stress such as in the interior of thick samples or at the lateral edges of a hole cut in a sample. I. M. Ward, “Mechanical Properties of Solid Polymers, 2'nd Ed.” Wiley, N.Y., 1983.

Degradation of the copolymers blends after extrusion or molding will also be examined. U.S. Pat. No. 6,794,485. For example, a molded sample such as stent can be used directly for the biodegradation test or the blended polymer may be cut into cubes after extrusion. Any desired shape or volume may be used for the test, ranging from about 0.5 mm³ to about 1 mm³, 10 mm³ to about 100 mm³, from about 20 mm³ to about 80 mm³, or from 40 mm³ to about 60 mm³. The polymer sample is then placed in a solution to study its degradation. In one embodiment, the sample is placed in phosphate buffer solution (PBS, pH 7.4) at 37° C. The physical properties of the polymer sample may be studied for about 1 month, 2 months, 3 months, 4 months, about 6 months and 1 year. The in vitro degradation kinetics of the present bioabsorbable polymers show less than about 5% overall breakdown after storage for 1 month at physiological conditions (e.g., phosphate buffered saline at 3° C.); in other embodiments, the overall breakdown is less than about 10%, 20%, 30% or 40% after storage for 1 month, 2 months, 3 months or 6 months at physiological conditions.The solution used for the degradation test may also be Tris-buffered saline (TBS), 4-(2-hydroxyethyl)-1-piperazineethanesulfonic acid (HEPES) buffer, 3-(N-morpholino)propanesulfonic acid (MOPS) buffer, piperazine-N,N′-bis(2-ethanesulfonic acid) (PIPES) buffer, or any other desired buffer system. The pH of the buffer may range from about 6 to about 8.5, from about 6.8 to about 8, or from 7.2 to about 7.6. The degradation test may be conducted at about 20° C. to about 50° C., from about 25° C. to about 45° C., about 47° C., or at about 37° C. The pH, composition and volume of the buffer system may remain the same or vary from the beginning to the end of the test period. The temperature at which the degradation test is conducted may remain the same or vary from the beginning to the end of the test period. Prior to the characterization of the polymer sample, it may be washed with distilled water and dried in a vacuum. The physical and mechanical properties of the polymer are assayed as described above. In one embodiment, the molecular weights of the polymers are measured by GPC. The degradation rates can be estimated by the mass loss (%) and molecular weight reduction (%). The polymer blend can also be examined by scanning electron microscope (SEM).

Degradation of polymers may also be examined using TOF-SIMS spectroscopy. U.S. Pat. Nos. 6,864,090 and 6,670,190. By tuning the biodegradable polymers of the present invention to degrade at a specific rate, drug elution can be precisely controlled and ceases entirely with the complete degradation of the polymer.

In addition, the degradation products are assayed for immunological properties by titering their effect on (i) Leukocyte Migration, (ii) Endothelial Cell Adhesion, (iii) Integrin-Mediated Adhesion, (iv) T cell proliferation, (v) B cell proliferation, (vi) T cell activation, (vii) COX Activity Assay, (viii) cytokine activation, (ix) Arachidonic Acid cascade, (x) Matrix Metalloproteinases, (xi) Signal transduction pathway activation, e.g., EGF, (xii) Transcription Factor, e.g., NFKB, and (xiii) growth factors, e.g., TGF.

The following examples are considered to be non-limiting and only representative of selected embodiments.

### EXAMPLE 1

Three batches of polymer blends were prepared. The compositions of the batches are shown below in table I.

TABLE I
Polymer Batches Compositions by Weight Percent

| | | | L-eCL³ | L-TMC⁴ | L-TMC |
|---|---|---|---|---|---|
| Batch | PLLA¹ | PDLA² | (70/30)⁵ | (80/20)⁶ | (70/30)⁷ |
| P-11369 | 33 | 47 | 20 | | |
| P-11371 | 40 | 40 | | 20 | |
| P-11228 | 33 | 47 | | | 20 |

¹Poly-L-lactide
²Poly-D-lactide
³Poly-L-lactide-co-ε-caprolactone
⁴Poly-L-lactide-co-TMC
⁵molar ratio L-lactide to -ε-caprolactone: note these molar rations only represent nominal ratios, i.e., the standard error is +/−5%
⁶nominal molar ratio L-lactide to TMC
⁷nominal molar ratio L-lactide to TMC

Differential scanning calorimetry (DSC) and Wide Angle Scattering X-ray difffraction (“WAXS”) was done on each sample.

The polymer blends were extruded into a long, hollow tube having varying wall thicknesses. In certain cases, the tubes were cut into ringlets having a width of 1-2 mm. Before analysis, the tubes or ringlet were disposed on an annealing mandrel having an outer diameter of equal to or less than the inner diameter of the tube and annealed at a temperature between about the polymer glass transition temperature and the melting temperature of the polymer blend for a time period ranging from about five minutes to 18 hours in air, an inert atmosphere or under vacuum. In various embodiments, the time of annealing ranged from about 5 minutes to about 2 hours, about 10 minutes to about 1 hour, about 15 minutes to about 30 minutes or about 15 minutes. The temperature of annealing ranged about 60° C. to about 150° C., from about 70° C. to about 140° C., from 80° C. to about 120° C. In the present example, P-11371 and P-11369 were annealed for 15 minutes at 80° C. and P-11228 was annealed for 15 minutes at 120° C.

In several cases, the tubes or ringlets were stressed after annealing by sliding the tube or ringlet on to a tapered mandrel having an outer diameter greater than the inner diameter of the tube or ringlet. The degree of expansion ranged from about 10% (d1/d2) to about 50% (d1/d2) where dl represents staring or initial diameter and d2 represents expanded diameter.

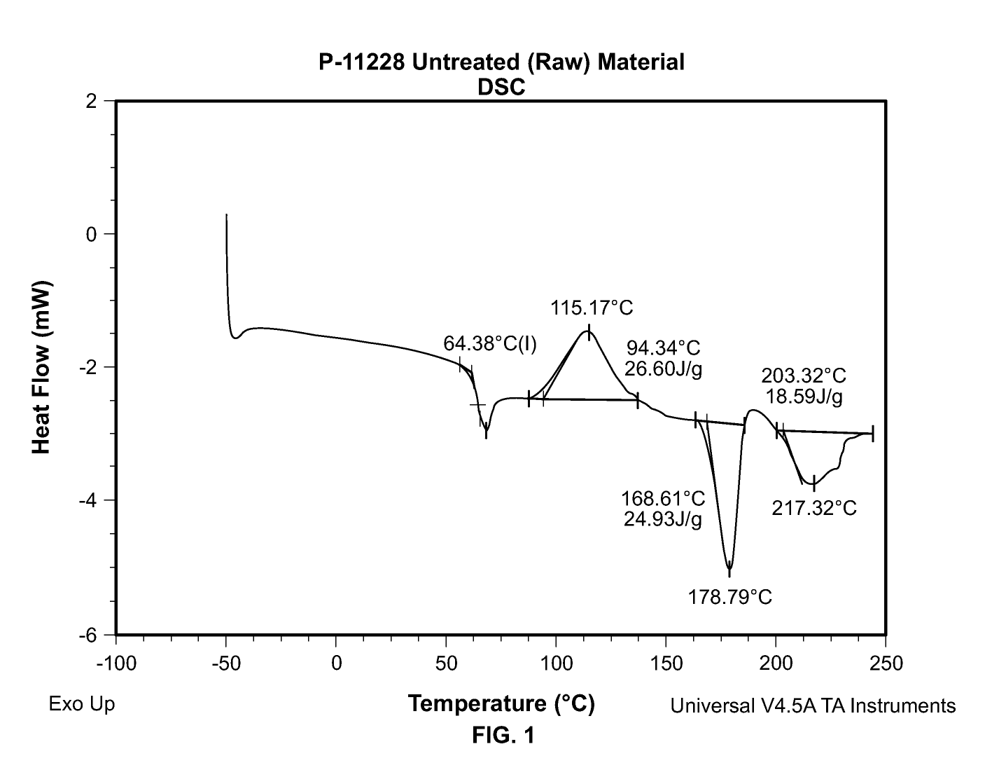

The DSC Thermograms for the batches are shown in FIGS. 1 through 9, P11228, P11369 and P11371. The DSC thermograms were produced using at TA Instrument Q10 DSC. Approximately 3 mg of each material was placed in an aluminum pan and sealed. The sample pan was placed into the DSC instrument with an empty aluminum pan as its reference. The material was then heated using a ramp program from −50 to 250° C. at 20° C./min. The TA Software was then used the calculate the approximate T_{g} , T_{c}, and Tₘ, if they occurred.

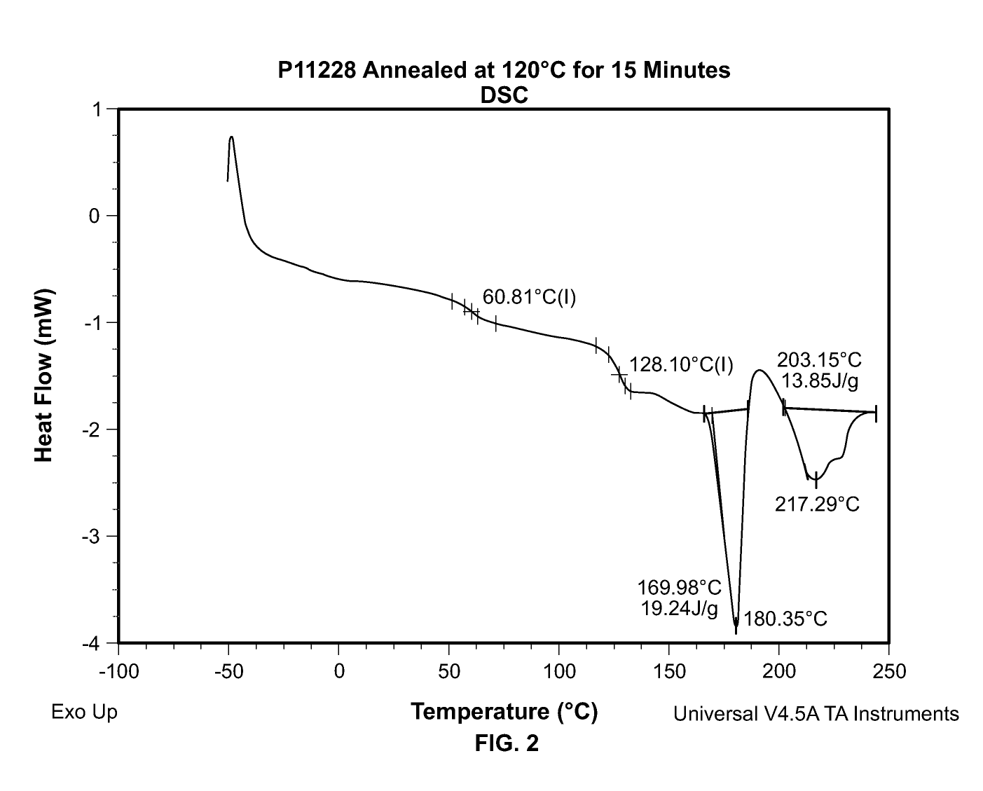

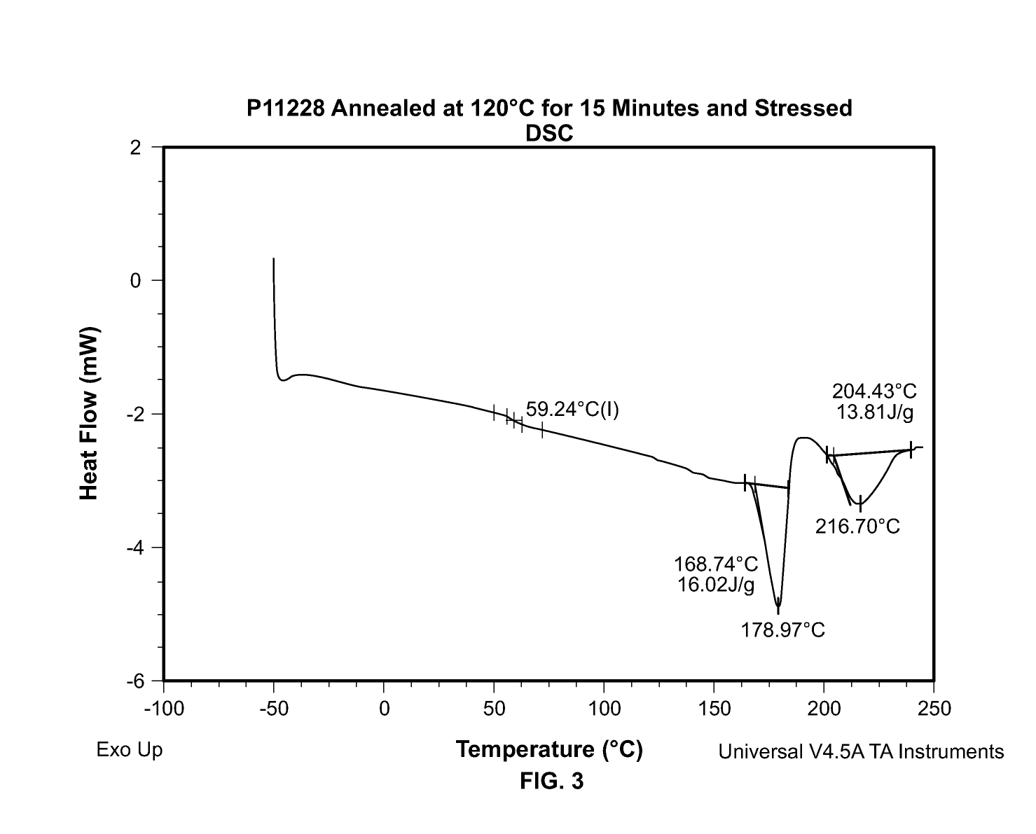

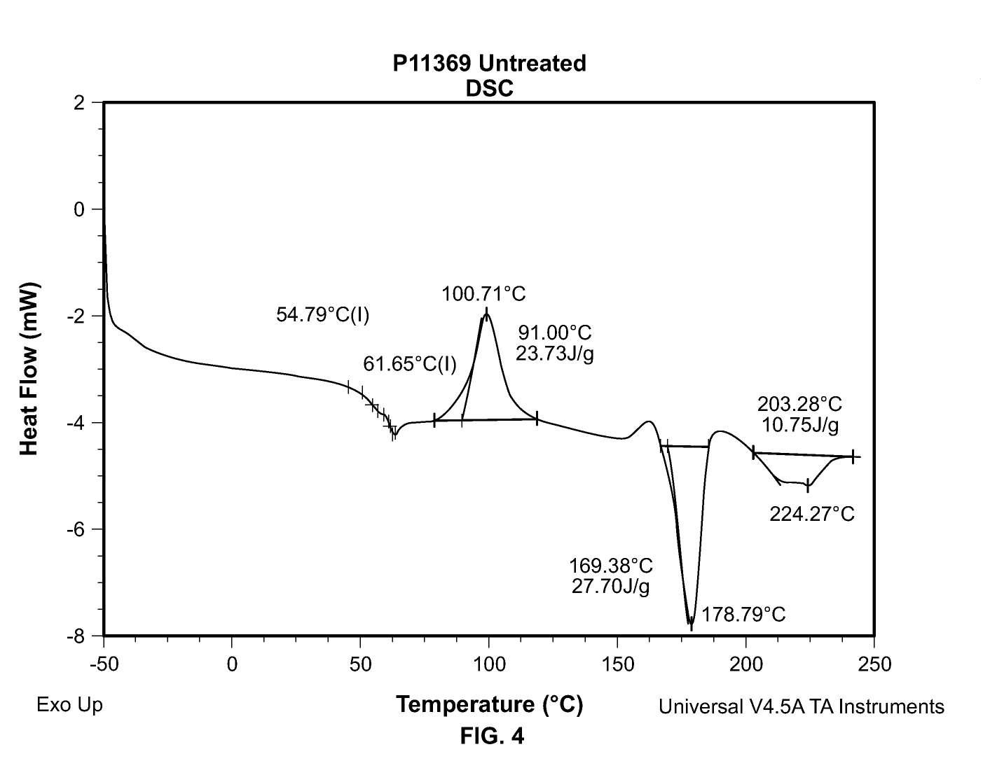

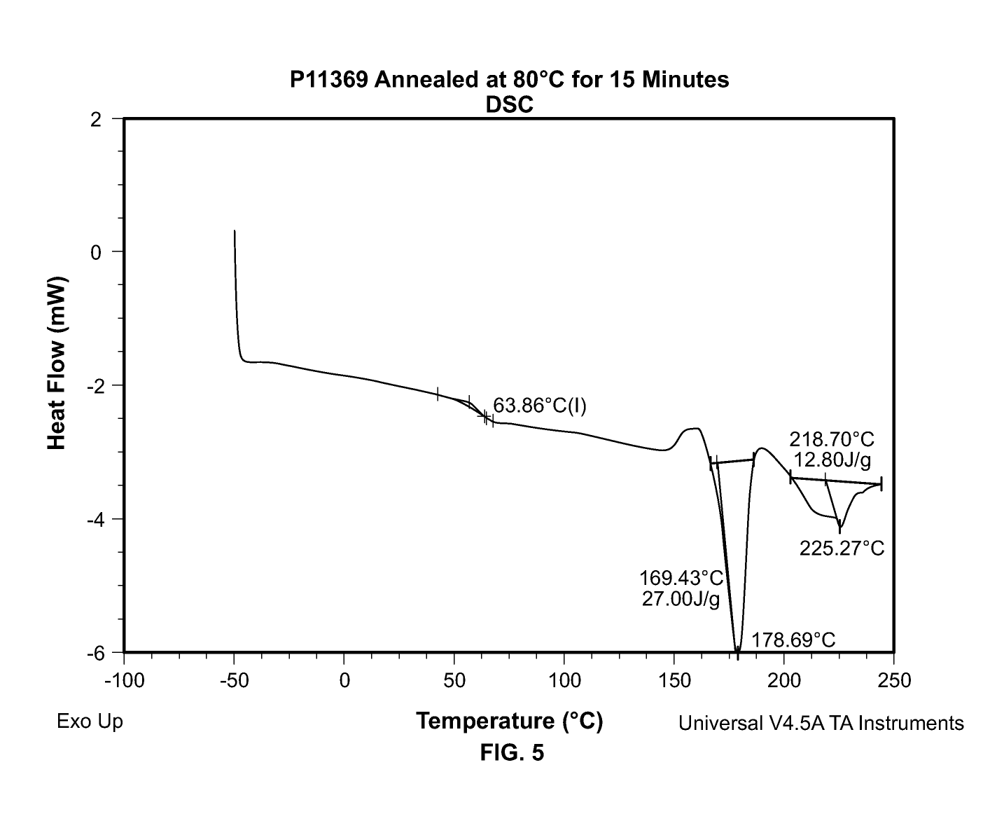

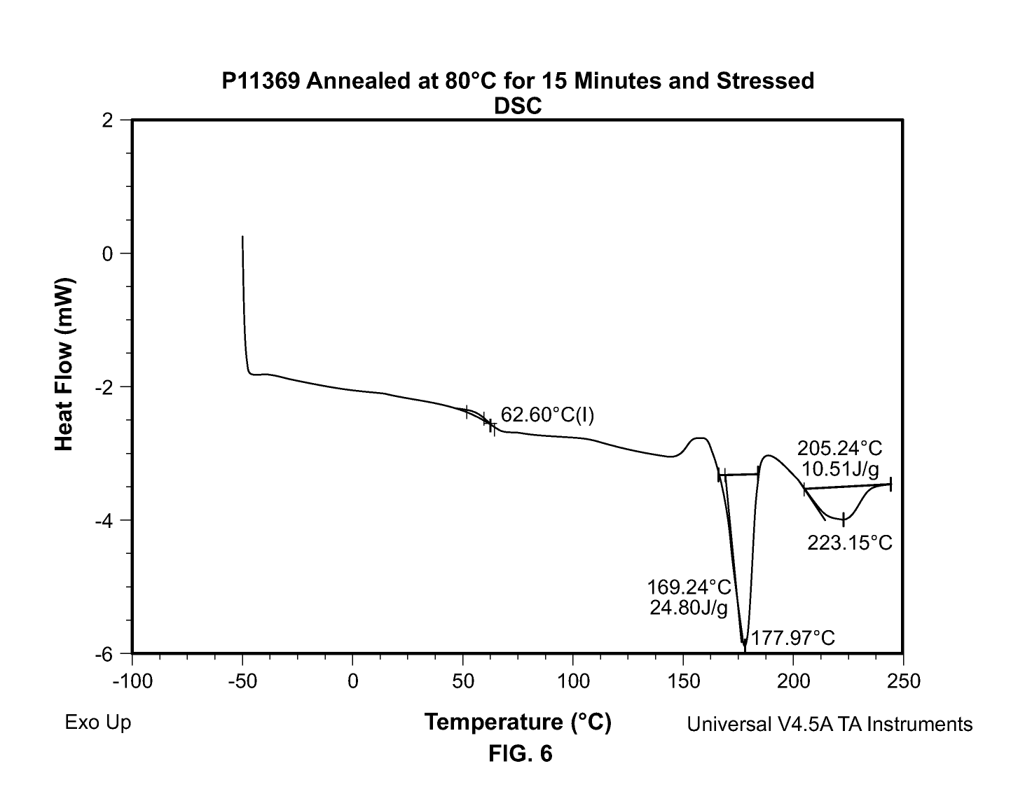

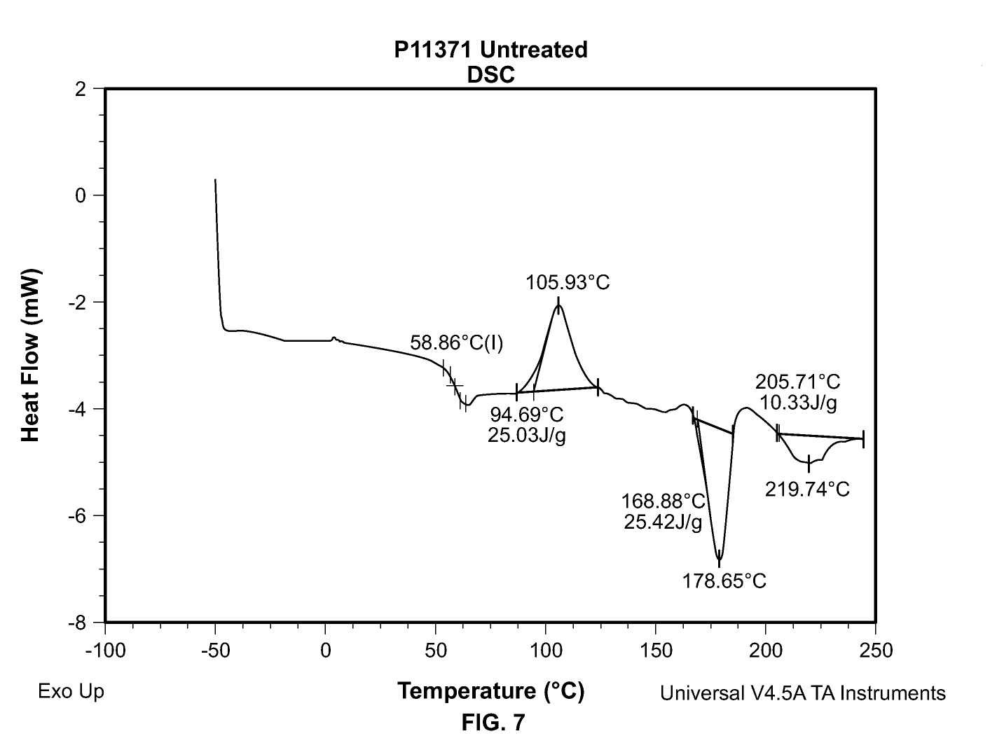

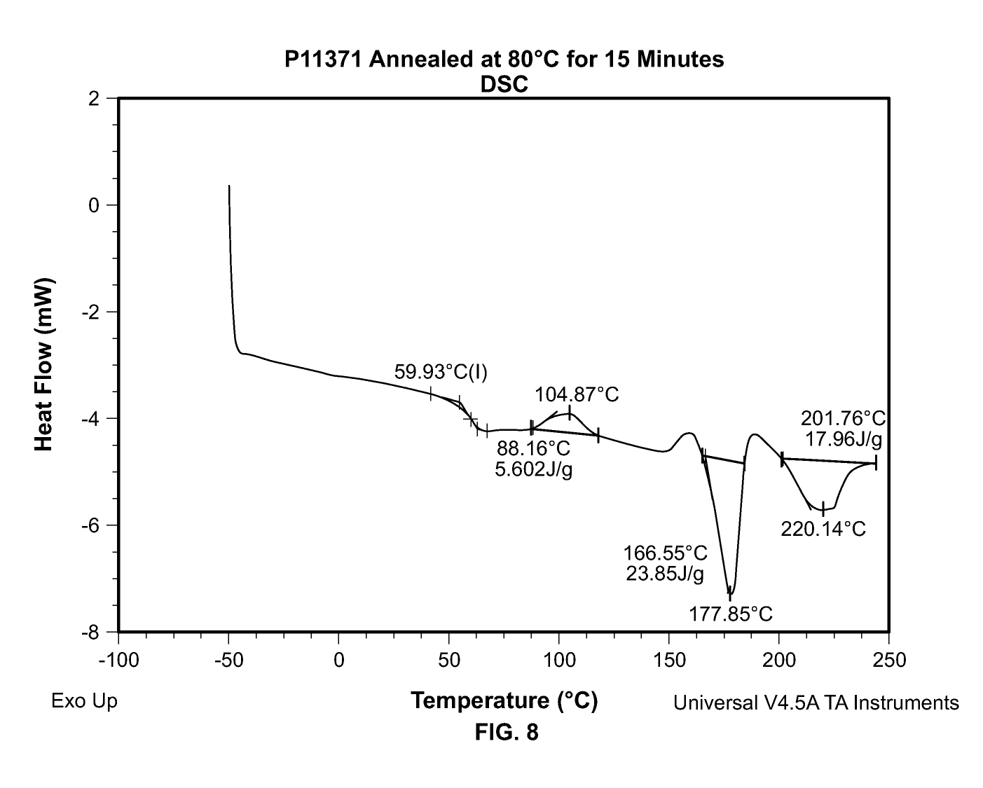

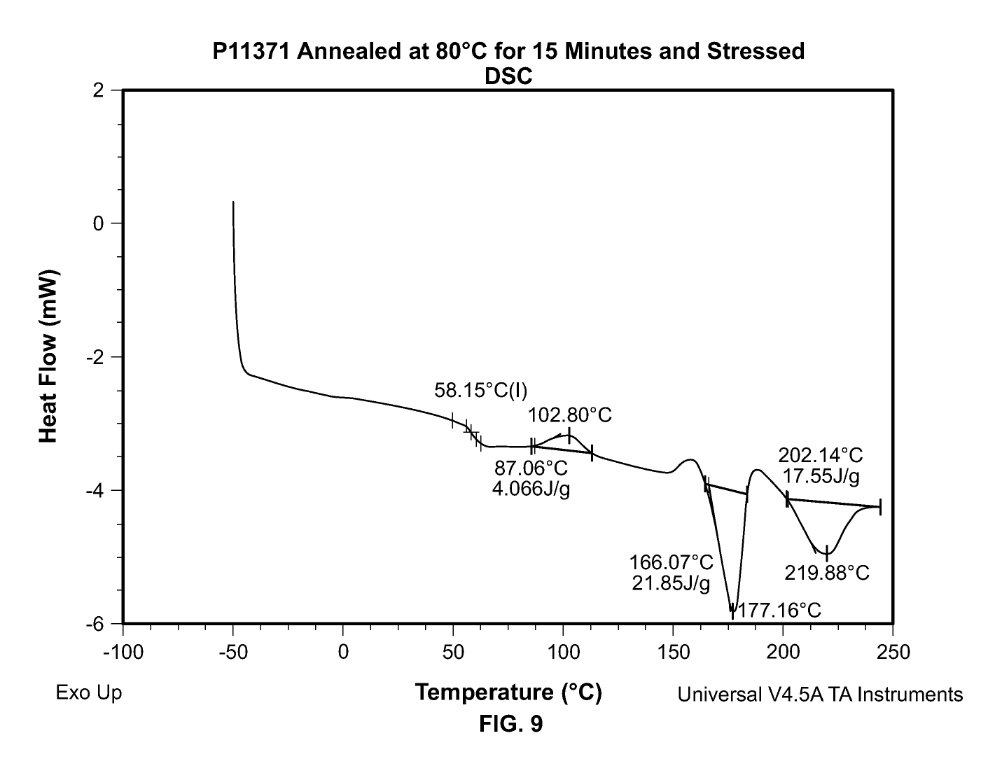

TABLE II
Summary of DSC Analysis

| | T_{g} | T_{c} | Tₘ¹ | ΔHₘ² | ΔH_{c}³ |
|---|---|---|---|---|---|
| FIG. 1 P11228-Raw | 64° C. | 115° C. | 179° C., | 43.5 J/gm | 26.6 J/gm |
| (Untreated) | | | 217° C. | (Joules/gram) | |
| FIG. 2 P11228-Annealed | 61° C., | | 180° C., | 33.1 | |
| | 128° C. | | 217° C. | | |
| FIG. 3 P11228-Annealed- | 59° C. | | 179° C., | 29.8 | |
| Stressed | | | 217° C. | | |
| FIG. 4 P11369-Raw | 55° C. | 100° C. | 179° C., | 38.5 | 23.7 |
| (Untreated) | | | 224° C. | | |
| FIG. 5 P11369-Annealed | 64° C. | | 179° C., | 39.8 | |
| | | | 225° C. | | |
| FIG. 6 P11369-Annealed- | 63° C. | | 178° C., | 35.3 | |
| Stressed | | | 223° C. | | |
| FIG. 7 P11371- | 59° C. | 106° C. | 179° C., | 35.7 | 25 |
| Raw(Untreated) | | | 220° C. | | |
| FIG. 8 P11371-Annealed | 60° C. | 105° C. | 178° C., | 41.9 | 5.6 |
| | | | 220° C. | | |
| FIG. 9 P11371- | 58° C. | 103° C. | 177° C., | 39.4 | 4.1 |
| Annealed-Stressed | | | 220° C. | | |

¹The Tₘ values represent approximate peak values with the lower value being the first or homoenatiomer crystalline structure which is melting and the upper value is the approximate peak of melting for the stereocomplex.
²The noted values are approximate.
³The noted values are approximate.

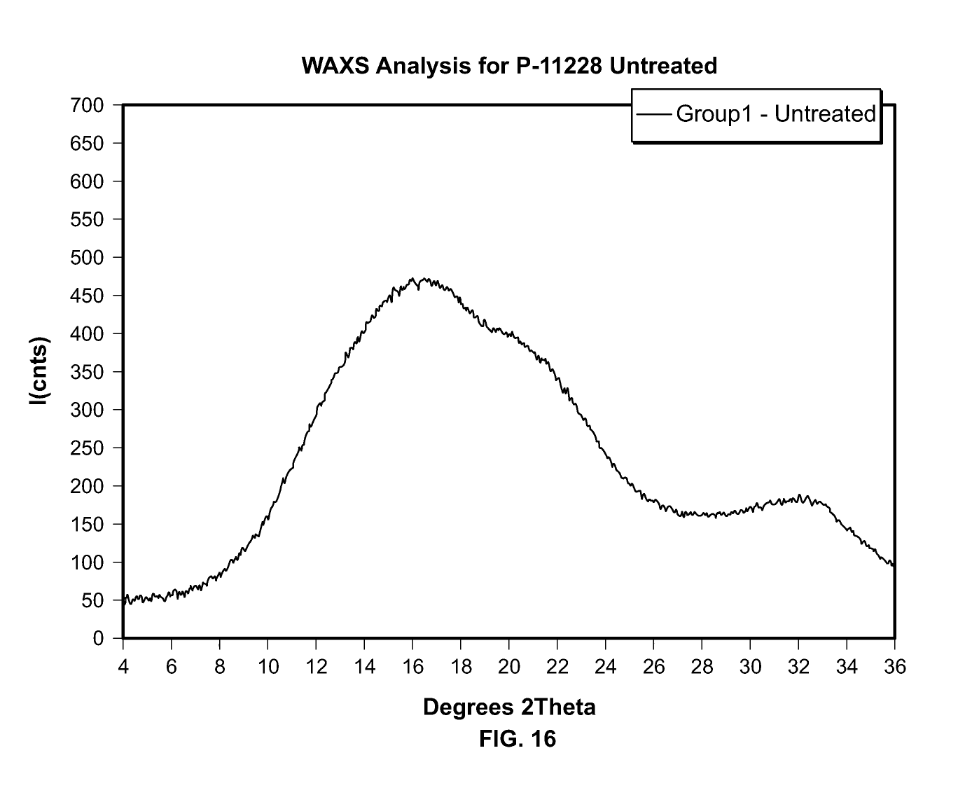

FIG. 1, P11228 untreated, presents a single strong T_{g} at about 64° C., a crystallization exotherm at about 115° C. with a H_{c} of about 26.6 J/g. There are 2 distinct Tₘ one peak at about 179° C. representing the homo-enantiomer crystal of poly-L or D Lactide and the other peak at about 217° C. representing the stereocomplex of L and D. The H_{c} at 115° C. does not offset the total Hₘ suggesting the presence of some crystallization in the raw or untreated state. However, the corresponding WAX (FIG. 16) shows the untreated sample as predominately amorphous. The heat of crystallization of the stereocomplex appears to be in the same temperature range as part of the homo-enantiomer melting curve masking or offsetting the exotherm.

FIG. 2, P11228 annealed, presents two glass transitions at about 61° C. and 128° C. The appearance of a T_{g} at 128° C. suggests a complex glass transition associated with the stereocomplex and significant domain differentiation between the stereocomplex and homo-enantiomer crystals. The absence of a crystallization exotherm at about 115° C. suggests that there is no crystallization occurring during the heating during the DSC test and that the associated dual crystal structures at 180° C. and 217° C. were produced during annealing.

FIG. 3, P11228 annealed and stressed presents only a single T_{g} at about 59° C. and two distinct Tₘ one at about 179° C. (representing the Poly-lactide homo-enantiomer crystal) and one at about 217° C. (representing the stereocomplex crystal). The absence of the second T_{g} at 128° C. (see, FIG. 2) suggests strain induced reordering into crystal morphology.

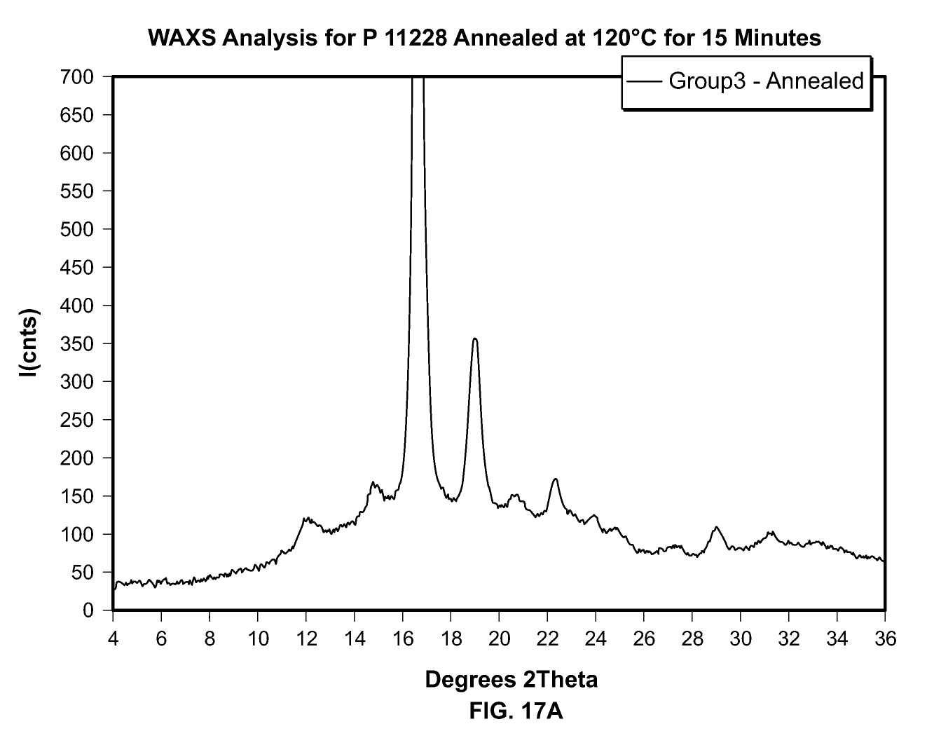

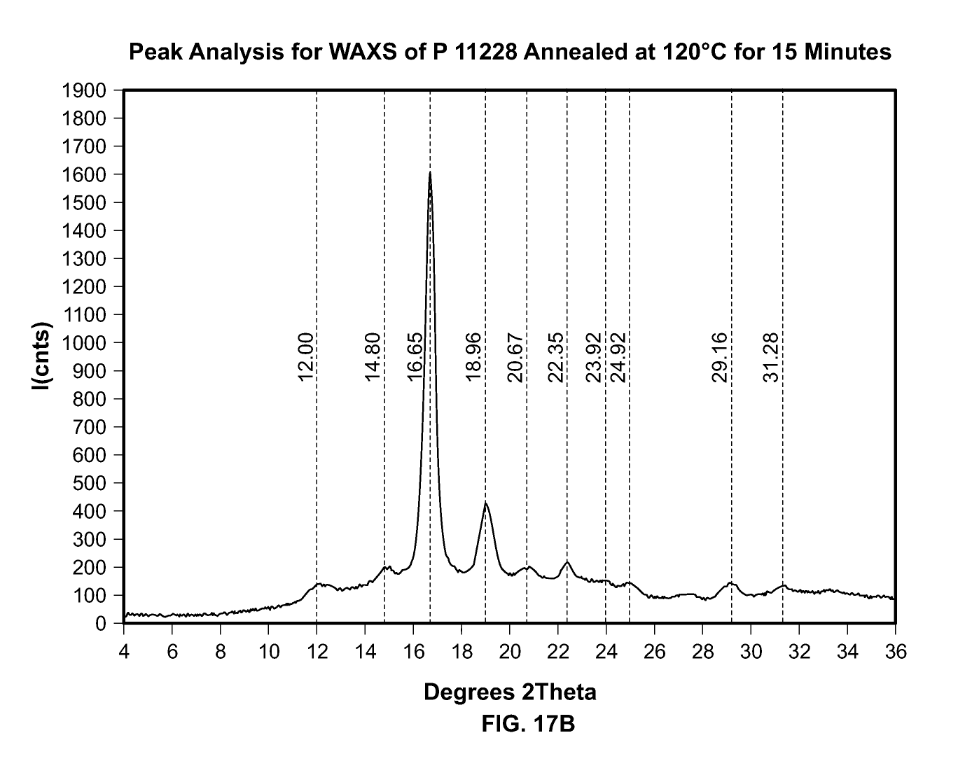

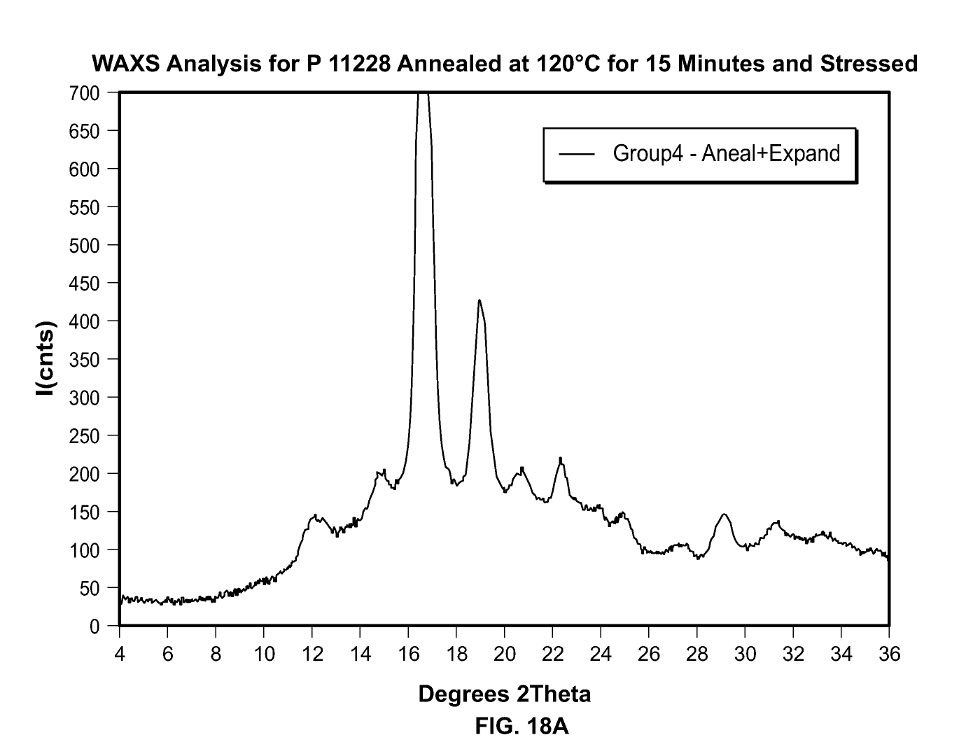

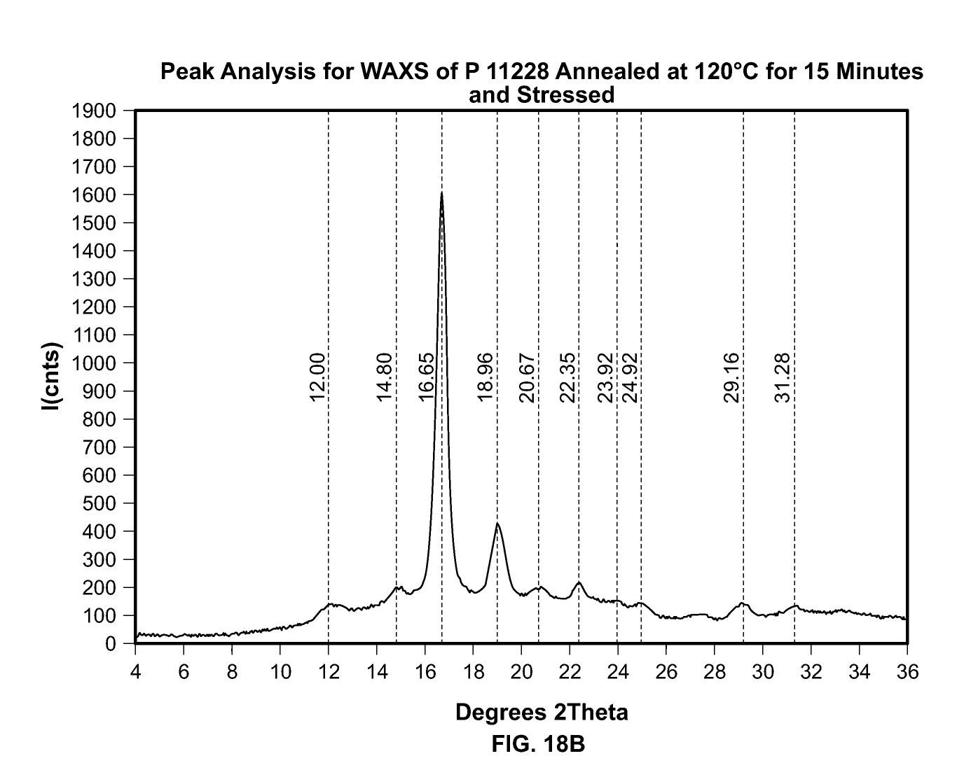

The corresponding WAXS patterns, see FIGS. 17a and b below confirms the coexistence of both the pseudo orthorhombic crystal structure of the poly-L or D-lactide homo-enantiomer crystal and the triclinic crystal of the polylactide stereocomplex as shown in the DSC (FIG. 2). After stressing, see, FIGS. 18a and b, below continues to show both L and/or D homo-enantiomer crystal morphology along with the stereocomplex. The peak width indicates an increase in crystallinity with the introduction of stressing the sample.

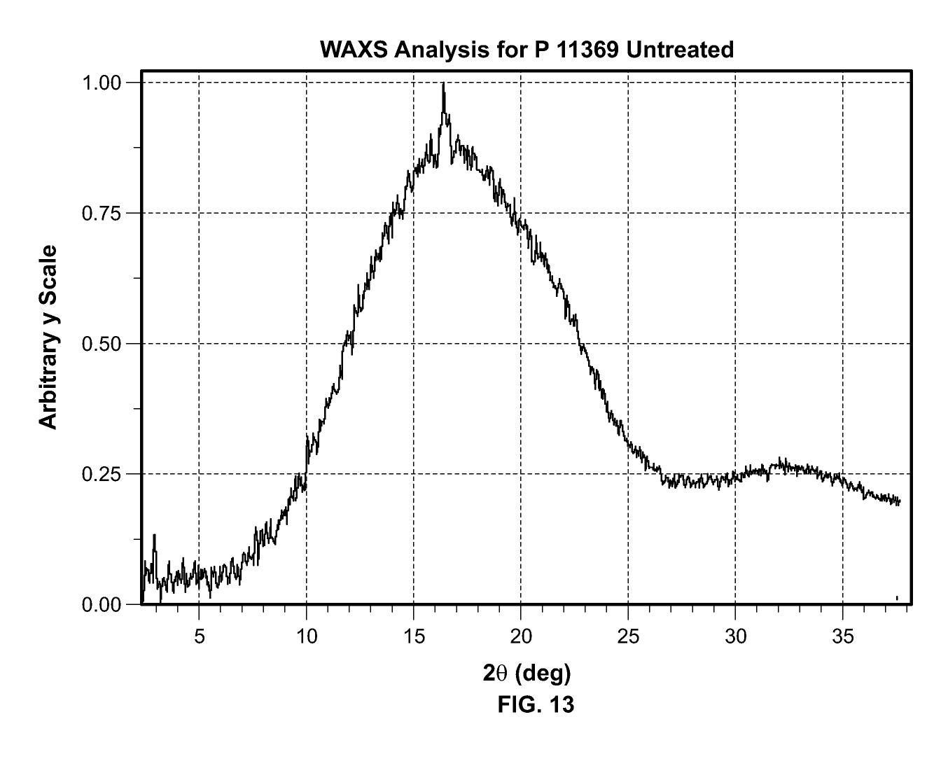

FIG. 4, DSC for P11369 untreated, presents a single T_{g} at about 55° C., a strong crystallization exotherm of about 23.7 J/g at about 100° C., and 2 distinct melting endotherms one at about 179° C. and at about 224° C. with a combined Hₘ of about 38.5 J/g. These two melting peaks correspond to the multiple crystal morphologies of the poly-L and/or D lactide homo-enantiomer and the polylactide stereocomplex. The H_{c} at about 100° C. of about 23.7 J/g does not appear to account for all of the crystal structure melting in the two subsequent endotherms, suggesting either the presence of some crystallinity in the untreated sample or unaccounted crystallization exotherm in the 195° C. region as discussed for FIG. 1. The corresponding WAX diffraction pattern for this sample (FIG. 13) confirms that the untreated sample is predominately amorphous.

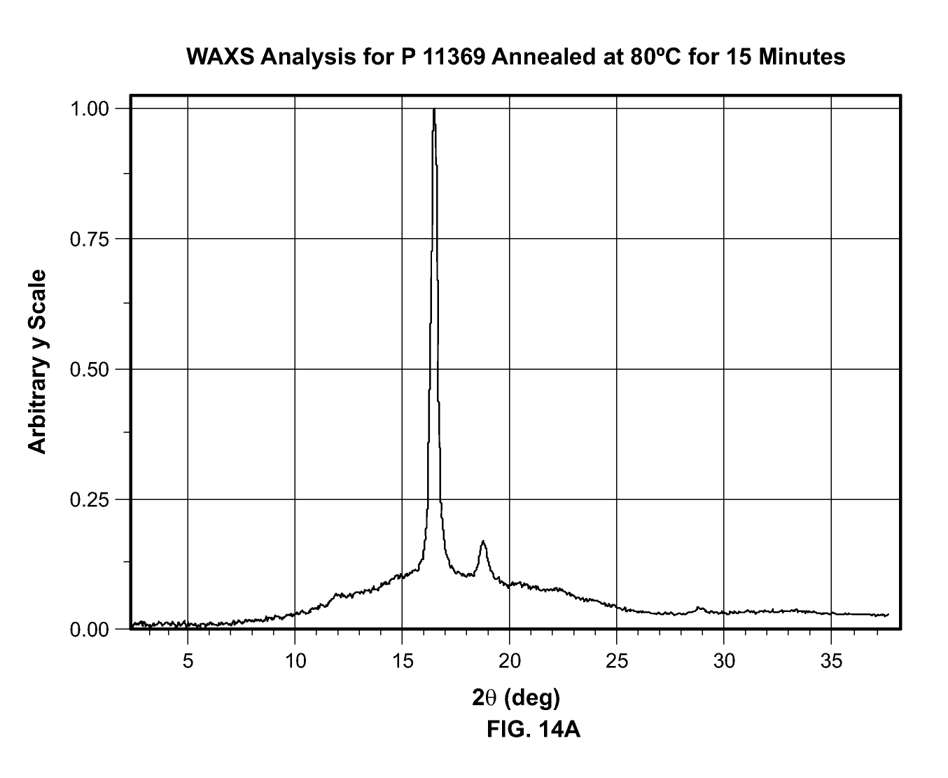

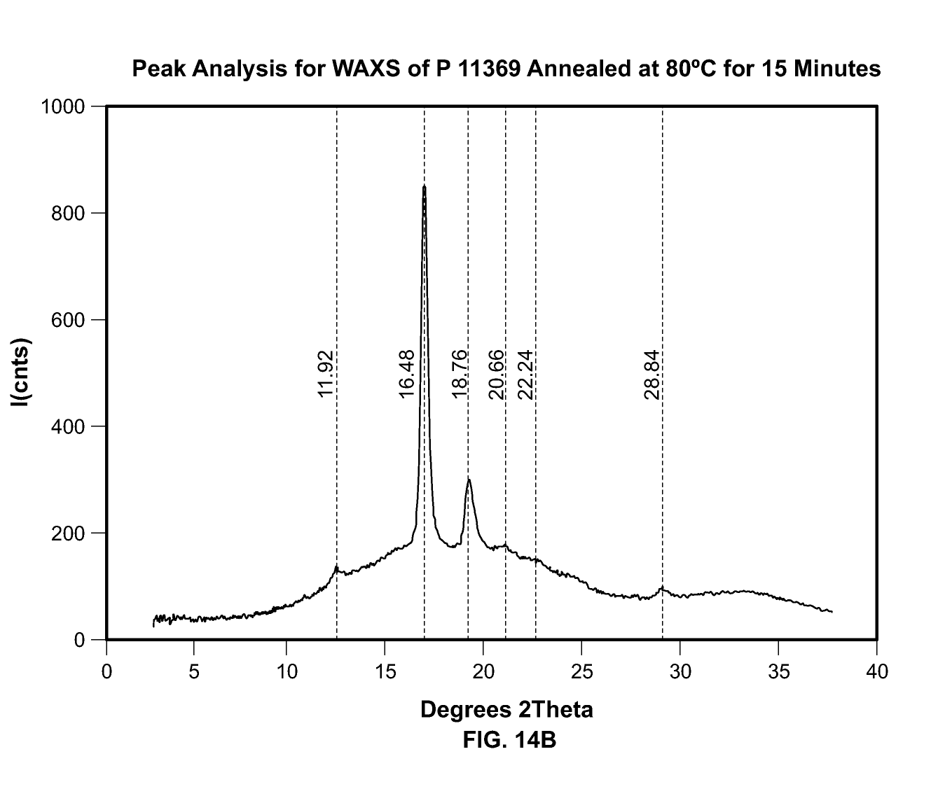

FIG. 5 which shows the DSC for P11369 annealed, shows a single strong T_{g} at about 64° C. and 2 distinct crystalline melting endotherms at about 179° C. and 225° C. corresponding to the poly-L and/or D lactide homo-enantiomer crystal and the polylactide stereocomplex crystal structures, respectively. The absence of the crystallization exotherm from FIG. 4 at about 100° C. suggests that the crystallization occurred during the annealing. The corresponding WAXS analysis, see, FIGS. 14a and b below shows the dominate crystal structure present being that of the D and/or L polylactide homo-enantiomer. This reveals that even though the DSC shows the stereocomplex in this sample, the formation of the stereocomplex appears to be suppressed at this annealing condition and is predominately formed during the DSC heating cycle.

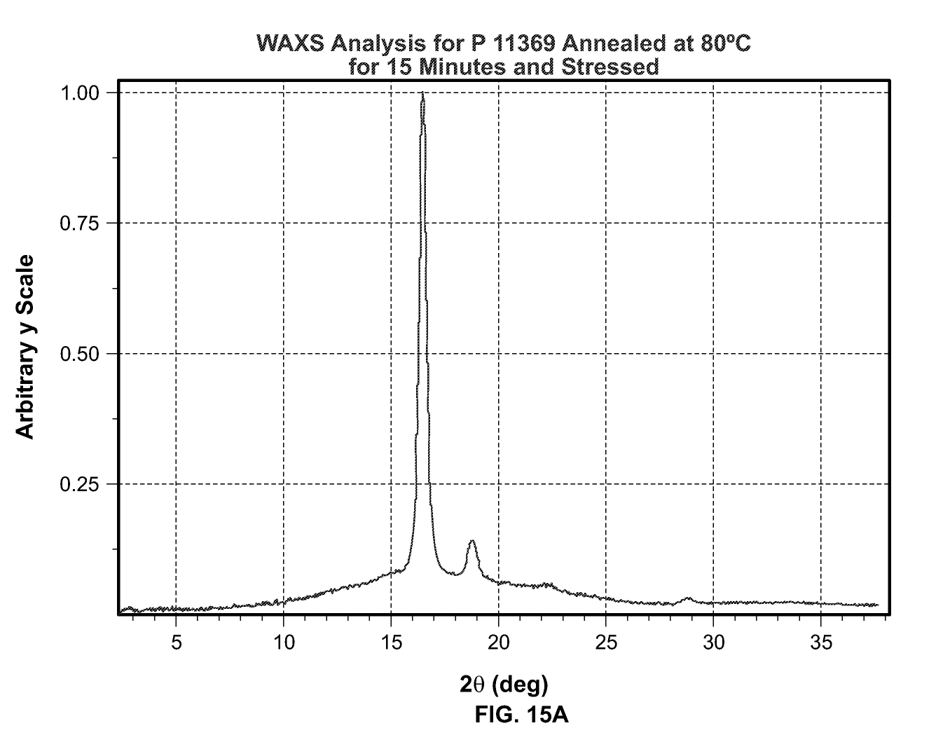

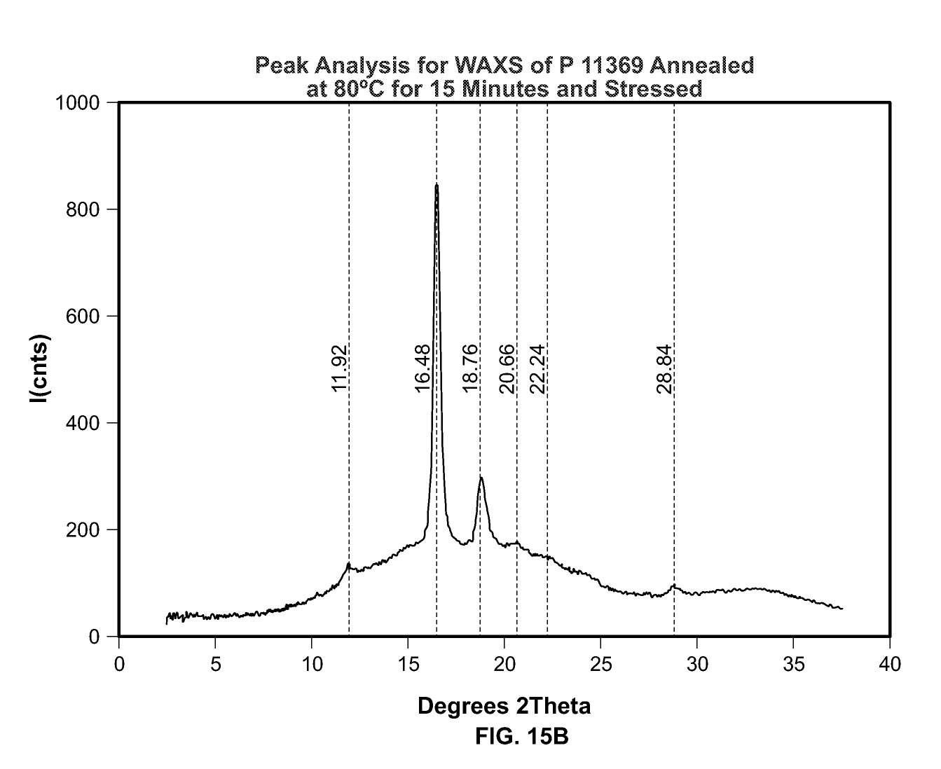

FIG. 6, DSC for P11369 annealed and stressed, shows a single T_{g} at about 63° C. and two strong crystalline melting endotherms at about 178° C. and 223° C. representing the poly L and/or D lactide homo-enantiomer and poly-lactide stereocomplex crystal morphologies. The corresponding WAXS analysis, see, FIGS. 15 a and b below, shows wider peaks representing an increase in degree of crystallization due to the applied stress. Further, the strain induced crystal morphology appears to remain unchanged from the unstressed sample.

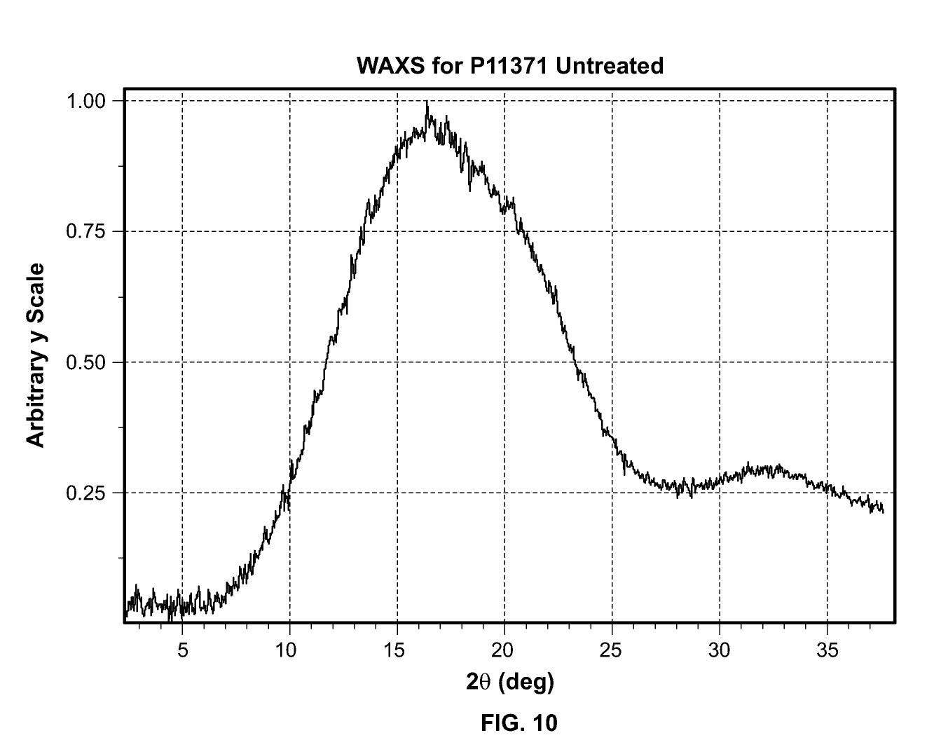

FIG. 7 shows the DSC for P11371 untreated. This DSC presents a strong T_{g} at about 59° C., and what appears to be a weak transition at below 0° C. suggesting a small degree of immiscibility. A significant crystallization exotherm of about 25 J/g presents at about 106° C. Two crystalline melting endotherms at about 179° C. and 220° C. represent the poly L and/or D lactide homo-enantiomer and polylactide stereocomplex crystal structures with a total Hₘ of about 35.7 J/g suggests the presence of some crystallinity in the untreated sample or unaccounted for crystallization exotherm for the stereocomplex at about 190° C. The corresponding WAX diffraction pattern for this sample (see, FIG. 10 below) confirms that the untreated sample is predominated amorphous.

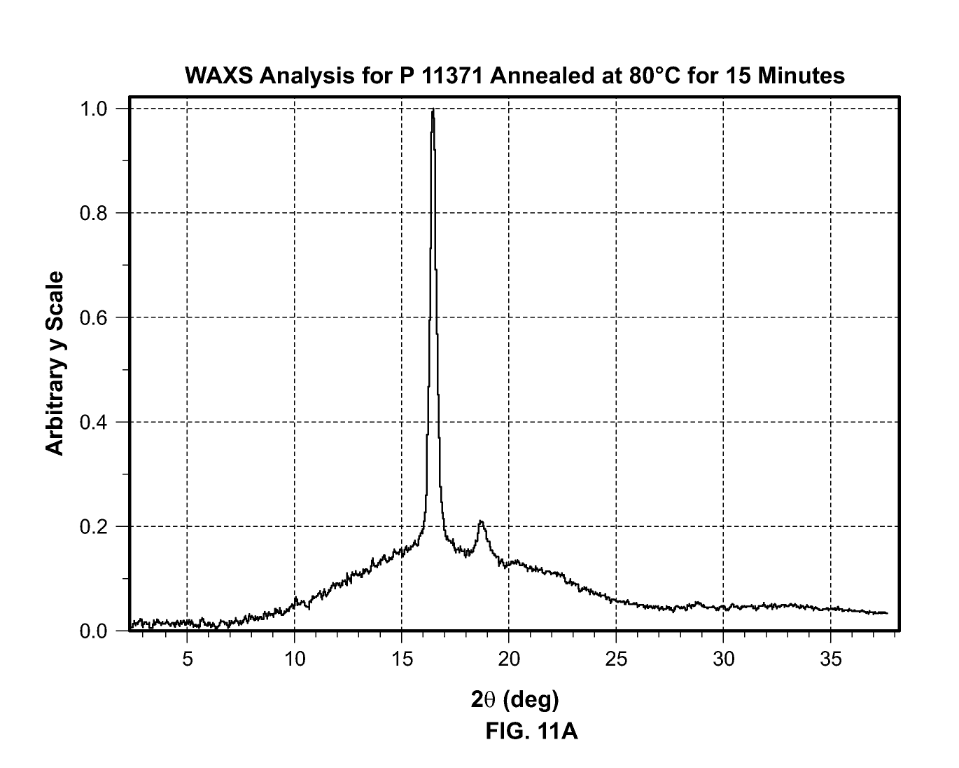

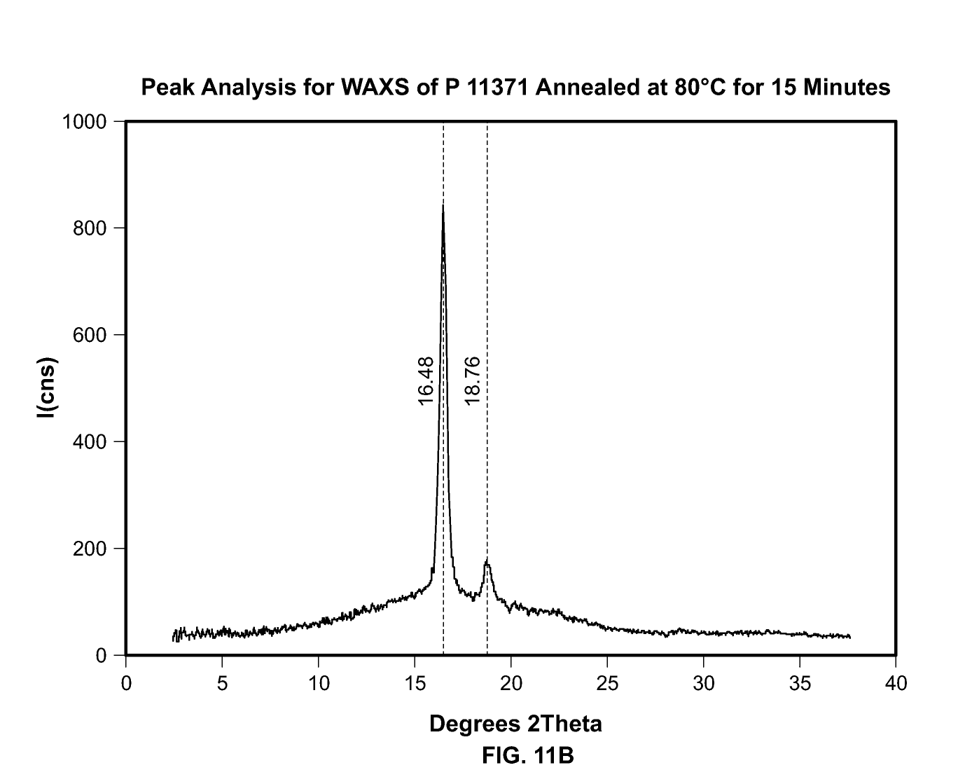

FIG. 8 shows the DSC for P11371 annealed. This DSC presents a single T_{g} at about 60° C., a small crystallization exotherm of about 5.6 J/g at about 105° C., and two distinct crystalline melting endotherms at about 178° C. and about 220° C. with a combined Hm of about 41.97J/g. The presence of the crystallization exotherm suggests that this annealing condition for this formulation leaves polymer that may be crystallized during the heat ramp cycle of the DSC, that is, remains available for further crystallization. The corresponding WAXS data, see, FIGS. 11 a and b, WAX for P11371 annealed show predominately the crystal morphology of the poly L and/or D polylactide homo-enantiomer. This reveals that even though the DSC shows the stereocomplex in this sample, the formation of the stereocomplex appears to be suppressed at this annealing condition and is predominately formed during the DSC heating cycle.

FIG. 9 shows the DSC for P-11371 annealed and stressed. This DSC presents a T_{g} at about 58° C., a small crystallization exotherm of about 4.1 J/g at about 103° C., and two distinct crystalline melting endotherms at about 177° C. and about 220° C. representing both the poly L and/or D lactide homo-enantiomer crystal as well as the poly-lactide stereocomplex. The somewhat smaller heat of crystallization presented in this DSC versus that of FIG. 8 suggests crystallization induced by the stress applied to the sample.

The samples were analyzed by x-ray diffraction. XRPD patterns were collected using a Bruker D-8 Discover diffractometer and Broker's General Detector System (GADDS, v. 4.1.20). An incident micro-beam of Cu Ka radiation was produced using a fine-focus tube (40 kV, 40 mA), a Gael mirror, and a 0.5 mm double-pinhole collimator. The incident X-ray optics are effectively “parallel beam”. With the use of an area detector system, there are no secondary X-ray optics between the sample and detector. Prior to the sample measurement, a silicon standard (NIST SRM 640c) was analyzed to verify the Si 111 peak position.

A specimen of the sample was supported using a capillary and secured to a translation stage. A video camera and laser were used to position the area of interest to intersect the incident X-ray beam in reflection geometry. When allowed by the sample geometry, some rocking of the sample was used during data collection to optimize orientation statistics. A beam-stop was positioned close to minimize air scatter from the incident beam.

Diffraction patterns were collected using a Hi-Star area detector located 15 cm from the sample and processed using GADDS. The detector and incident X-ray beam are not moved during the active data collection period and the area detector returns a 2D image of the powder diffraction rings produced by the sample. The intensity in the GADDS image of the diffraction pattern was integrated using a step size of 0.04° 2θ over the range 2.0 to 37.6° 2θ. The integrated patterns display diffraction intensity as a function of 2θ. The absolute error in 20 (x-axis) is about +/−0.2 degrees, while the relative error (peak to peak differentiation) is about +/−0.02. The error in the peak intensity is about 5% (see, H. P. Klug and L. E. Alexander: X-ray Diffraction Procedures For Polycrystalline and Amorphous Materials: Wiley-Interscience Publication, 1974 (second edition)). Table III presents the WAXS data.

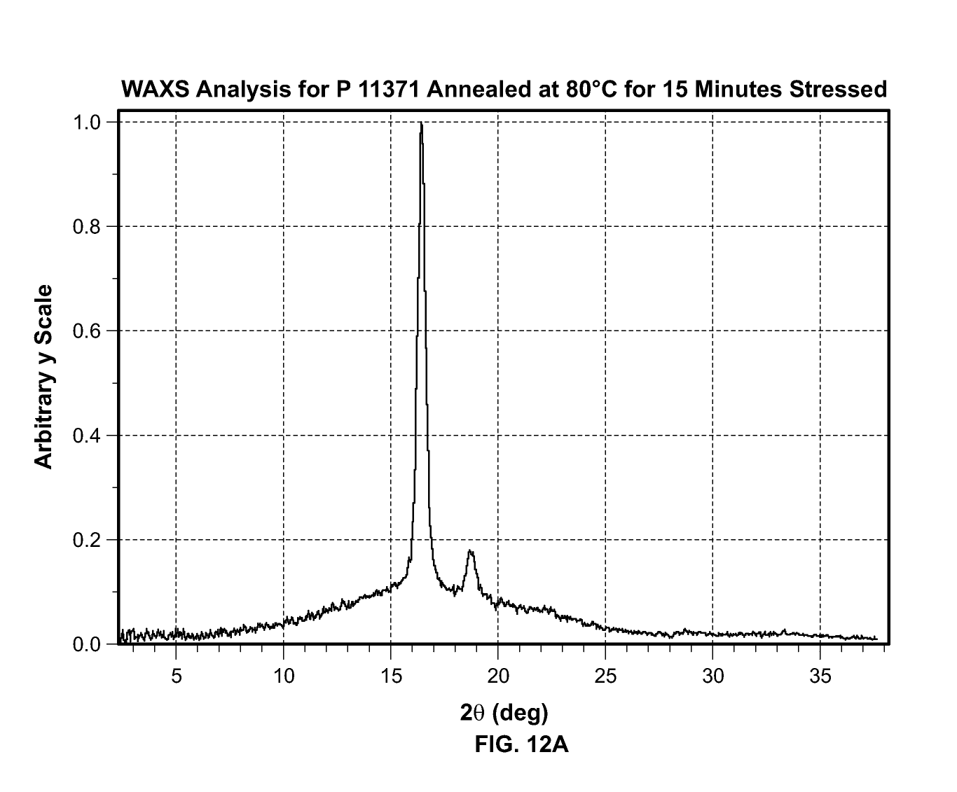

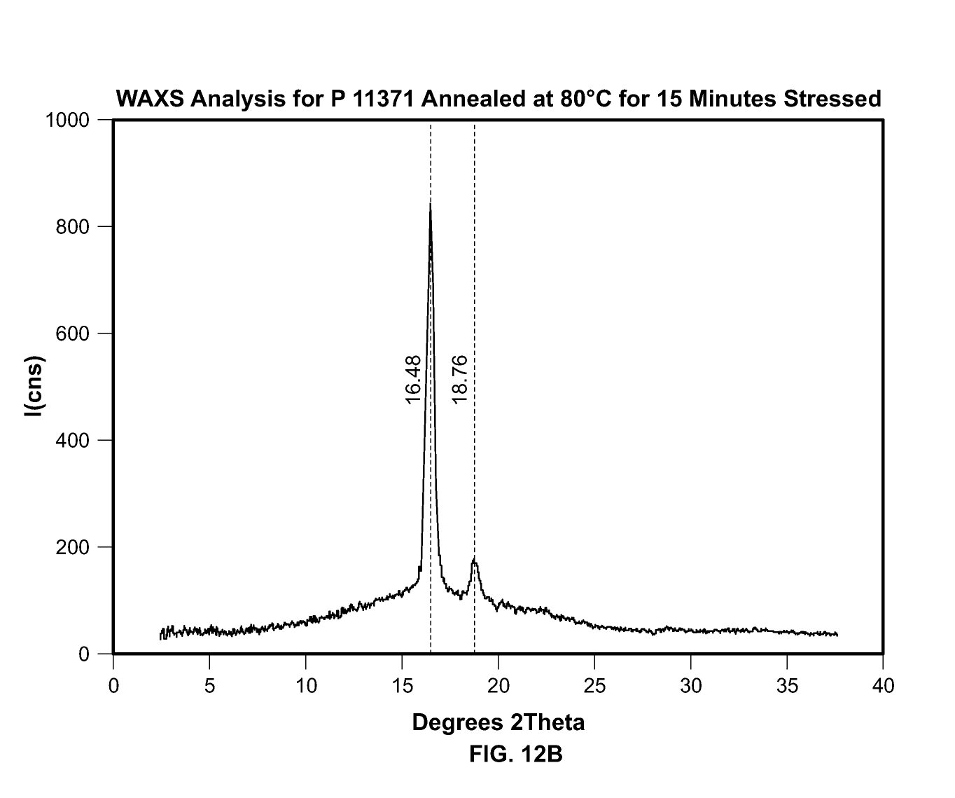

TABLE III
WAXS Analysis Summary

| | 2θ Peaks |
|---|---|
| FIG. 10 P11371-Raw | Amorphous |
| FIG. 11 a, b P11371-Annealed | 16.48, 18.76 |
| FIG. 12 a, b P11371- | 16.48, 18.76 |
| Annealed-Stressed | |
| FIG. 13 P11369-Raw | Amorphous |
| FIG. 14 a, b P11369-Annealed | 11.92, 16.48, 18.76, 20.66, 22.24, |
| | 28.84 |
| FIG. 15 P11369-Annealed- | 11.92, 16.48, 18.76, 20.66, 22.24, |
| Stressed | 28.84 |
| FIG. 16 P11228-Raw | Amorphous |
| FIG. 17 a, b P11228-Annealed | 12.00, 14.80, 16.65, 18.96, 20.67, |
| | 22.35, 23.92, 24.92, 29.16, 31.28 |
| FIG. 18 a, b P11228-Annealed- | 12.00, 14.80, 16.65, 18.96, 20.67, |
| Stressed | 22.35, 23.92, 24.92, 29.16, 31.28 |

FIG. 10 shows the X-ray powder diffraction pattern taken from an intact tube of raw or unprocessed material (P11371). The sample appeared amorphous. i.e., no crystallinity was observed for this sample. The sensitivity of the WAXS machine is capable of detecting 1% or greater crystalline material in the sample. Amorphous material indicates that overall crystallinity was less than about 95% (w/w), less than about 98% (w/w) or less than about 99% (w/w).

FIGS. 11 a and b (diffraction peaks identified) shows the X-ray powder diffraction pattern taken from an intact annealed tube of material (P 11371). A large crystalline response on an amorphous halo corresponding to about 23.4% crystallinity was observed. The width of the main crystalline peak (pseudo Voight) is about 0.352 degrees.

FIG. 12 a and b (diffraction peaks identified) shows the X-ray powder diffraction pattern taken from intact ringlet material that was annealed and stressed (P 11371). Stressing was caused by sliding material over a tapered mandrel, similar to that seen in the DSC data. A large crystalline response on an amorphous halo corresponding to about 36.5% crystallinity was observed. The width of the main crystalline peak (pseudo Voight) is about 0.418 degrees.

FIG. 13 shows the X-ray powder diffraction pattern taken from an intact tube of raw or unprocessed material (P11369). The X-ray powder diffraction pattern is predominately amorphous with a small crystalline peak at 16.5 2θ corresponding to about 1.0% crystallinity was observed. FIGS. 14 a and b (diffraction peaks identified) show the X-ray powder diffraction pattern taken from an intact annealed tube of material (P11369). A large crystalline response on an amorphous halo corresponding to about 29.5% crystallinity was observed. The width of the main crystalline peak (pseudo Voight) is about 0.367 degrees. The width of the main crystalline peak (pseudo Voight) is about 0.352 degrees.

FIGS. 15 a and b (diffraction peaks identified) show the X-ray powder diffraction pattern taken from intact, ringlet material that was annealed and stressed (P11369). A large crystalline response on an amorphous halo corresponding to about 35.7% crystalline was observed. The width of the main crystalline peak (pseudo Voight) is about 0.388 degrees.

FIGS. 16, 17 a and b (diffraction peaks identified) and 18 a and b (diffraction peaks identified) show the WAXS pattern for Batch P11228 under the conditions noted in the figures. Both the WAXS and corresponding DSC patterns show the presence of psuedo orthorhombic-crystals of the polyL or D-lactide homo-enantiomer crystals together with triclinic crystals of the lactide sterocomplex.

Table IV summarizes the percent crystallinity in each particular state for the two batches, P11369 and P11371.

TABLE IV
Percent Crystallinity

| | | | | % Change |
|---|---|---|---|---|
| | | | Annealed- | Annealed/Annealed- |
| Batch | Raw | Annealed | Stressed | Stressed |
| P-11369 | 1 | 29.5 | 35.7 | 21 |
| P-11371 | 0 | 23.4 | 36.5 | 56 |

Table IV shows the peak width for the various samples under several different conditions. Crystalline diffraction peak widths are good measure of the kinetic perfection of a crystalline material and can be used to characterize a materials micro-structure in terms of the size of perfect crystalline regions and micro-strain between the crystalline regions. Lanford et al., Powder Diffraction, Rep. Prog. Phys. 59:131-234 (1996).

TABLE V
Peak Width

| Batch | Crystallinity (%) | Peak Width (°) |
|---|---|---|
| P11369 - Annealed | 29.5 | 0.367 |
| P11369 | 35.7 | 0.388 |
| Annealed + Stressed | | |
| P11371 | 23.4 | 0.352 |
| Annealed | | |
| P11371 | 36.5 | 0.418 |
| Annealed + Stressed | | |

Batches, P11369 and P11371, were also tested for tensile strength and ductility. Tensile strength is the stress at the maximum on the engineering stress-strain curve and ductility is the measure of the degree of plastic deformation that has been sustained at fracture and can be expressed quantitatively as percent elongation, % EL=(1_{f}−10/10)×100.

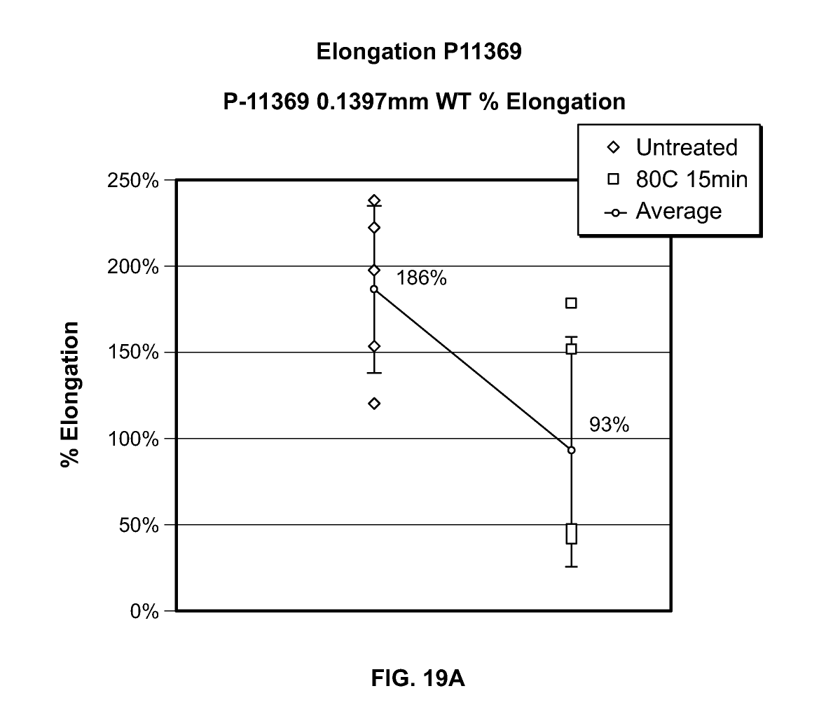

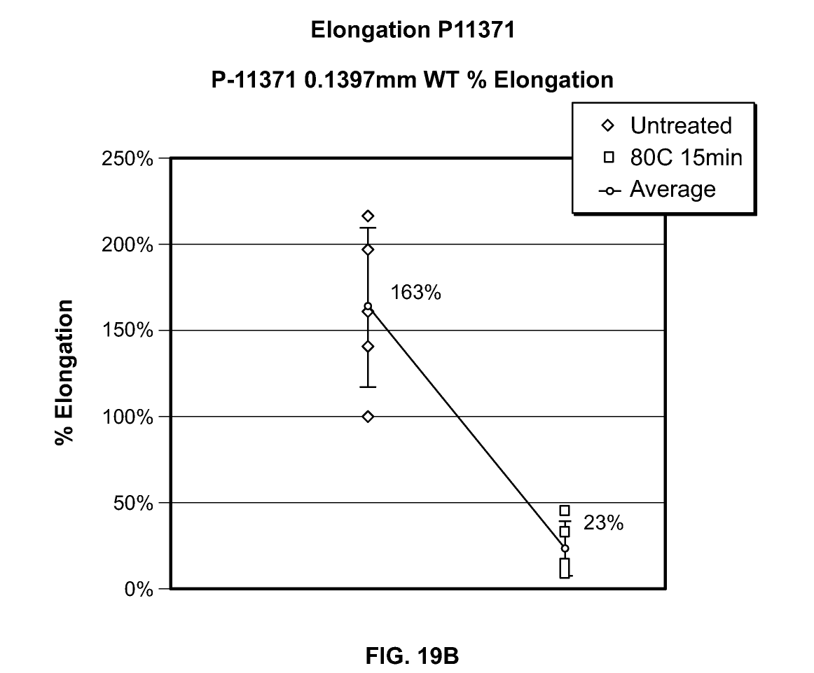

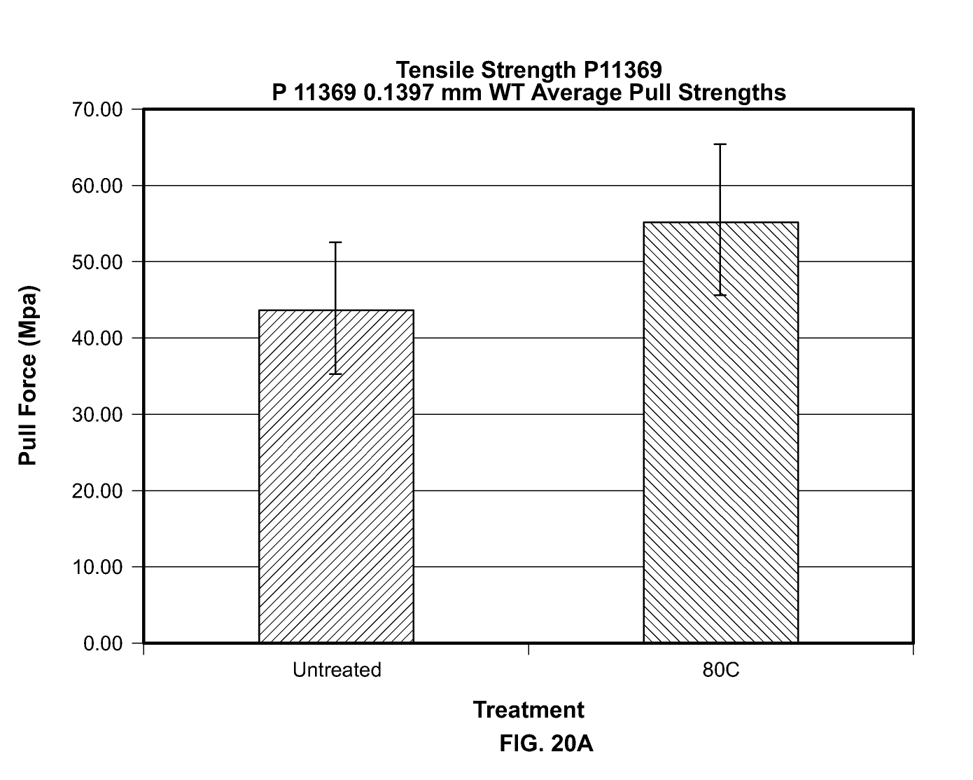

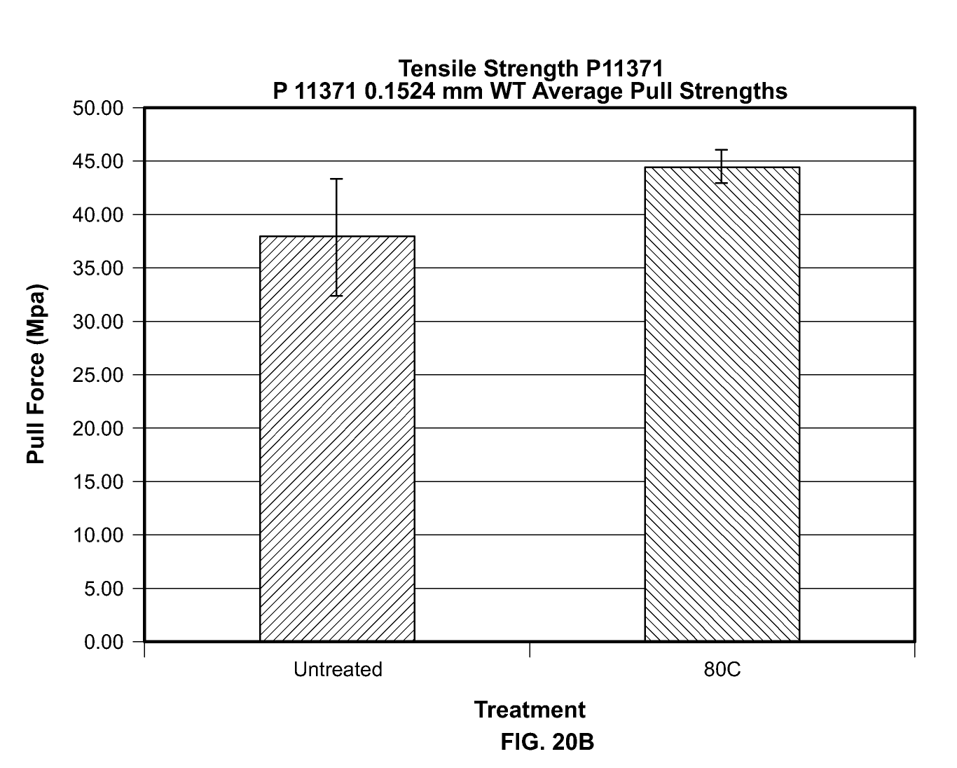

The tests were conducted as follows. A United Pull Test Fixture, Model # SSTM-1. United 51b Load Cell, Model # 5LB T was used. The samples cut into 1-2 mm sections and then loaded on ‘U’ shaped test wires, with the sections fixed between an upper clamp an lower clamp. The samples were lowered into a water bath at physiological temperatures and pulled for various times at about 4.7″/min. After pulling the samples were removed from the clamps and measured on a calibrated Micro-Vu. FIGS. 19a,b shows the results of the elongation analysis and FIGS. 20a,b the tensile or pull strength. The mean percent elongation for untreated P11369 is 186%+/−49%, while the mean percent elongation for P11369 which had been annealed at 80° C. for 15 minutes is 93%+/−67%; the mean percent elongation for untreated P11371 is 163%+/−46%, while the mean percent elongation for

P11371 which had been at 80° C. for 15 minutes is 23%+/−16%. The mean tensile strength for untreated P11369 is 43.81+/−8.6 (units are MegaPascals “MPa”), while the mean tensile strength for P11369 which had been annealed at 80° C. for 15 minutes is 54.88+/−10.97 MPa; the mean tensile strength for untreated P11371 is 37.89+/−5.44 MPa, while the mean tensile strength for P11371 which had been at 80° C. for 15 minutes is 44.88+/−1.62 MPa.

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

All patents, applications, publications, test methods, literature, and other materials cited herein are hereby incorporated by reference.

What is claimed is:

1. A composition comprising a blend formed from poly-L-lactide, poly-D-lactide or mixtures thereof and a copolymer moiety comprising poly-L-lactide or poly-D-lactide linked with β-caprolactone or trimethylcarbonate wherein, the poly-L-lactide or poly-D-lactide sequence in the copolymer moiety is random with respect to the distribution of ε-caprolactone or trimethylcarbonate and where the wide-angle X-ray scattering (WAXS) exhibits 20 values of about 16.48 and about 18.76.

2. The composition of claim 1 wherein the co-polymer moiety comprises poly-L-lactide or poly-D-lactide linked with ε-caprolactone.

3. The composition of claim 2 wherein the polymer moiety comprises poly-L-lactide.

4. The composition of claim 2 wherein the polymer moiety comprises poly-D-lactide.

5. The composition of claim 1 wherein the co-polymer moiety is poly-L-lactide or poly-D-lactide linked with TMC and the molecular weight of the co-polymer ranges from about 1.2 IV to about 2.6 IV.

6. The composition of claim 2 wherein the molecular weight of the co-polymer ranges from about 0.8 to about 6.0.

7. The composition of claim 1 wherein the WAXS 2θ values further comprise peaks at about 11.92, about 20.66, about 22.24 and about 28.84.

8. The composition of claim 1 comprising a blend having about 20%-45% (w/w) poly-L-lactide, about 35% (w/w) to about 50% (w/w) poly-D-lactide and about 10% (w/w) to about 35% (w/w) poly L-lactide-co-TMC or poly-L-lactide-ε-caprolactone.

9. The composition of claim 1 wherein the poly-L-lactide or poly-D-lactide ranges from about 20% (w/w) to about 95% (w/w).

10. The composition of claim 9 wherein the poly-L-lactide or poly-D-lactide ranges from about 50% (w/w) to about 95% (w/w).

11. The composition of claim 10 wherein the poly-L-lactide ranges from about 60% (w/w) to about 95% (w/w).

12. The composition of claim 11 wherein the poly-L-lactide ranges from about 70% (w/w) to about 80% (w/w).

13. The composition of claim 1 wherein greater than 7 L-lactides or D-lactides are arrayed sequentially in the copolymer moiety.

14. A composition comprising a blend formed from poly-L-lactide, poly-D-lactide or mixtures thereof and a copolymer moiety comprising poly-L-lactide or poly-D-lactide linked with ε-caprolactone or trimethylcarbonate wherein, the poly-L-lactide or poly-D-lactide sequence in the copolymer moiety is random with respect to the distribution of ε-caprolactone or trimethylcarbonate, wherein there is at least about 95% (w/w) amorphous material in the composition.

15. The composition of claim 14 wherein there is at least about 98% (w/w) amorphous material.

16. The composition of claim 15 wherein there is at least about 99% (w/w) amorphous material.

17. The composition of claim 1 wherein percent crystallinity ranges from about 0% (w/w) to about 10% (w/w).

18. The composition of claim 1 wherein the percent crystallinity ranges from about 20% (w/w) to about 70% (w/w).

19. The composition of claim 18 wherein the percent crystallinity ranges from about 30% (w/w) to about 60% (w/w).

20. The composition of claim 19 wherein the percent crystallinity ranges from about 30% (w/w) to about 60% (w/w).

21. A composition comprising a blend formed from poly-L-lactide, poly-D-lactide or mixtures thereof and a copolymer moiety comprising poly-L-lactide or poly-D-lactide linked with ε-caprolactone or trimethylcarbonate wherein, the poly-L-lactide or poly-D-lactide sequence in the copolymer moiety is random with respect to the distribution of ε-caprolactone or trimethylcarbonate and where the wide-angle X-ray scattering (WAXS) exhibits 20 values of about 16.65 and about 18.96.

22. The composition of claim 21 wherein the WAXS 20 values further comprise about 12.00, about 14.80, about 20.67, about 22.35, about 23.92, about 24.92, about 29.16 and about 31.28.

23. The composition of claim 1 wherein the Tₘ peaks occur at about 180° C. and about 217° C.

24. The composition of claim 21 wherein the Tₘ peaks occur at about 178° C. and about 220° C.

25. The composition of claim 21 wherein about T_{g} is 61° C. and about 128° C.
